(19) United States
(12) Patent Application Publication
(10) Pub. No.: US 2018/0118699 A1
(43) Pub. Date: May 3, 2018

(54) SK AND IK CHANNEL AGONISTS FOR TREATMENT OF HEART FAILURE

(71) Applicant: THE TRUSTEES OF COLUMBIA UNIVERSITY IN THE CITY OF NEW YORK, New York, NY (US)

(72) Inventors: Steven MARX, Scarsdale, NY (US); Elaine WAN, Fresh Meadows, NY (US); Donald LANDRY, New York, NY (US); Sergey SAKHAROV, Fort Lee, NJ (US); Guoxia LIU, New York, NY (US); Shi-Xian DENG, White Plains, NY (US); Xiaoming XU, New York, NY (US)

(21) Appl. No.: 15/819,732

(22) Filed: Nov. 21, 2017

Related U.S. Application Data
(63) Continuation-in-part of application No. PCT/US2016/033584, filed on May 20, 2016.
(60) Provisional application No. 62/165,628, filed on May 22, 2015.

(51) Int. Cl. C07D 277/84; A61K 31/428; A61K 45/06; A61P 9/04; A61P 9/12; A61P 3/10

(52) U.S. Cl. CPC C07D 277/84; A61K 31/428; A61K 45/06; A61K 2300/00; A61P 9/12; A61P 3/10; A61P 9/04

(57) ABSTRACT

The present invention provides compounds that are improved potassium channel agonists. Pharmaceutical compositions including a pharmaceutically acceptable carrier and a compound of the present invention are also provided. Methods and kits for treating or ameliorating the effects of heart failure syndrome, high blood pressure and diabetes also are provided. Methods, kits and compositions which include compounds of the present invention also are provided.

### CROSS-REFERENCE TO RELATED APPLICATIONS

This patent application is a continuation in part of PCT international application no. PCT/US2016/033584, filed May 20, 2016, which claims the benefit of U.S. Provisional Patent Application No. 62/165,628, filed May 22, 2015, which applications are incorporated by reference herein in their entireties.

### FIELD OF INVENTION

The present invention provides, inter alia, SK and IK channel activators and compositions containing such compounds. Methods for using such compounds or compositions are also provided.

### BACKGROUND OF THE INVENTION

In the initial phases of the heart failure (HF) syndrome, small resistance vessels, especially nonessential circulations such as the renal, splanchnic (e.g. mesenteric) and cutaneous vascular beds, constrict in order to maintain systemic blood pressure. For patients with HF, the body's adaptation can be more detrimental than the initial insult. As the HF syndrome progresses, the elevated vascular tone in coronary arteries and peripheral vessels becomes excessive and maladaptive, increasing cardiac workload, reducing myocardial perfusion, and predisposing the heart to ischemia (Zelis et al. 1982, Katz et al, 1992, Schrier et al. 1999). The reduction in myocardial blood flow and the increased cardiac workload conspire to impair cardiac output and ventricular remodeling, accelerating the progression to decompensated HF (Mroz et al. 2012, Duvvuri et al. 2012, He et al. 2011, Smith et al 2012). The increase in vascular tone reflects high levels of circulating vasoactive hormones and cytokines, formation of reactive oxygen species (ROS), and endothelial dysfunction. The endothelial dysfunction is manifested by reduced endothelial-mediated dilatation caused by diminished bioavailability of nitric oxide (NO) and endothelial-derived hyperpolarizing factors (EDHF) (Katz et al. 1992). Reducing this excessive vascular tone is an important therapeutic goal in the treatment of patients with HF, but in many cases the drugs, which do not directly target the molecules responsible for the dysfunction, lack efficacy and/or have side effects that can detrimentally affect cardiac function.

An unappreciated contributor to the increased vasoconstriction in HF is the intrinsic dysfunction of vascular smooth muscle (VSM) cells within resistance vessels. In mice with HF, VSM cells have abnormal electrical properties, namely reduced expression and activity of voltage- and Ca²⁺-activated large conductance K⁺ (BK) channels, increased depolarization of the membrane potential, and increased intracellular Ca²⁺ concentration, leading to enhanced pressure-induced vasoconstriction (myogenic tone) (Wan et al, 2013). Mice with deletion of the smooth muscle-specific BK β1 regulatory subunit also have markedly reduced survival and worsened HF after ligation of their left coronary artery (Brenner et al. 2000, Kumar et al. 2005). Without the BK β1 subunit, small vessels are hypercontractile in response to increases in luminal pressure and norepinephrine, the latter being markedly elevated after myocardial infarction (MI) and in HF (Xu et al. 2011, Graham et al. 2004, McAlpine et al. 1988, Sigurdsson et al. 1993, Lymperopoulos et al. 2010). The phenotype of decreased survival and cardiac function in BK β1 null mice is thus likely due to the failure to blunt coronary and/or peripheral vasoconstriction. This hypothesis offers an explanation for the increased incidence and severity of MI and HF in animals and humans with diabetes and hypertension. Diabetes and hypertension have been shown in most, but not all animal models to be associated with reduced expression and/or function of the pore-forming BK α subunit and its β1 regulatory subunit (Rusch 2009, Phillips et al. 2005, Dong et al. 2009, Liu et al. 2009, Nieves-Cintron et al, 2008, Amberg et al, 2003(a), Amberg et al. 2003(b), Bagi et al. 2005, Lagaud et al 2001, McGahon et al. 2007(a), McGahon et al. 2007(b), Lu et al. 2008, Dong et al. 2008, Yang et al. 2012).

An important approach to treat HF is to reduce vascular resistance, but most therapies do not specifically target the intrinsic dysfunctions within endothelial and VSM cells. Nitrates can reduce vascular contractility, but the benefits of nitrates, shown in animal and human studies, are short-lived due to the development of tolerance and pseudotolerance (Gupta et al. 2013). Since BK channels play a central role in the mediation of the vasodilator response to NO and other nitrates, the findings of their reduced expression and function provide a mechanism to explain, at least in part, the limited effectiveness of nitrates (Bychkov et al. 1998). Many other vasodilators either decrease cardiac contractility (e.g. Ca²⁺ channel antagonists), which preclude their use in patients with HF, or can increase heart rate, arrhythmias and long-term mortality (e.g. dobutamine, milrinone). BK channels are especially attractive targets because of their critical function in the peripheral vasculature, their putative cardioprotective role in mitochondria of cardiomyocytes, and their absence in the plasma membrane of cardiomyocytes (Wan et al. 2013, Babu et al. 2007, Clements et al. 2011, Singh et al, 2013). These characteristics are particularly important for designing treatments for HF patients.

The present invention is provided to overcome, inter alia, the challenges noted above.

### SUMMARY OF THE INVENTION

Drugs that enhance endothelial derived hyperpolarizing factor (EDHF), an alternative pathway that is both not dependent upon the function of BK channels and not affected by tolerance, may represent a new class of agents for HF patients. Activation of endothelial Ca²⁺-activated small and intermediate conductance K⁺ channels (SK3 and IK1) can indirectly hyperpolarize the underlying VSM cells. Whereas endothelial-derived vasodilators, such as P450-derived epoxyeicosatrienoic acids (EET), NO, prostacyclin, lipoxygenase products and hydrogen peroxide, hyperpolarize and relax VSM cells by activating BK channels, BK channels are not required for EDHF-mediated vasodilation. Increasing SK3 and IK1 currents by transgenic or pharmacological approaches decreased myogenic tone, increased acetylcholine-induced relaxation of rat cremaster arterioles, and restored the attenuated EDHF-type relaxation in mesenteric arteries from Zucker diabetic rats. Activating SK3 and IK1 channels induced dilation and increased coronary flow in Langendorff-perfused rat hearts. SKA-31, a specific activator of SK3 and IK1 channels, caused dilation of mesenteric resistance vessels isolated from mice with HF. The present invention describes improved SK and IK agonist compounds which are shown herein to activate heterologously expressed SK3 channels.

One embodiment of the present invention is a compound having formula (I):

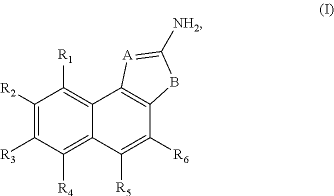

wherein
A and B are independently selected from the group consisting of S or N;
R₁, R₂, R₃, R₄, and R₆ are independently selected from the group consisting of H, halogen, alkyl, ester, ether, thioether, aryl, heteroaryl, CN, NO₂, and amine;
R₅, is selected from the group consisting of H, ester, thioether, aryl, heteroaryl, NO₂, and amine;
wherein each alkyl is optionally substituted with a group consisting of halide, ether, and combinations thereof;
wherein each aryl and each heteroaryl are optionally substituted with a group consisting of halide, ether, C₁₋₄alkyl, and combinations thereof; and
wherein each amine is optionally substituted with a group selected from halide, C₁₋₄alkyl, and combinations thereof,
   and crystalline forms, hydrates, or pharmaceutically acceptable salts thereof,
   with the provisio that the compound is not

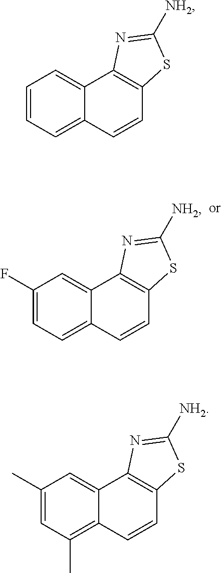 [SMILES: Nc1nc2c(ccc3ccccc32)s1 ; Nc1nc2c(ccc3ccc(F)cc32)s1 ; Cc1cc(C)c2ccc3sc(N)nc3c2c1]

Another embodiment of the present invention is a compound having the structure:

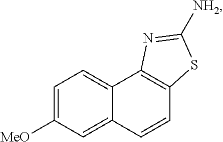 [SMILES: COc1ccc2c(ccc3sc(N)nc32)c1]

or a crystalline form, hydrate, or pharmaceutically acceptable salt thereof.

Another embodiment of the present invention is a compound having the structure:

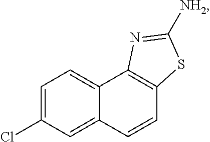 [SMILES: Nc1nc2c(ccc3cc(Cl)ccc32)s1]

or a crystalline form, hydrate, or pharmaceutically acceptable salt thereof.

Another embodiment of the present invention is a pharmaceutical composition comprising a pharmaceutically acceptable carrier and a compound of the present invention.

Another embodiment of the present invention is a method for treating or ameliorating the effects of a condition in a subject in need thereof comprising administering to the subject an effective amount of a compound of the present invention or a pharmaceutical composition of the present invention.

Another embodiment of the present invention is a method for treating or ameliorating the effects of heart failure syndrome (HF) in a subject in need thereof comprising administering to the subject an effective amount of a pharmaceutical composition of the invention.

Another embodiment of the present invention is a kit for treating or ameliorating the effects of a condition in a subject in need thereof, the kit comprising an effective amount of a compound of the present invention or a pharmaceutical composition of the present invention, packaged together with instructions for its use.

Another embodiment of the present invention is a kit for treating or ameliorating the effects of heart failure syndrome (HF) in a subject in need thereof, the kit comprising an effective amount of a pharmaceutical composition of the present invention, packaged together with instructions for its use.

Another embodiment of the present invention is a composition comprising a compound of the present invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIGS. 1A and 1B present graphs demonstrating the activity of KCa potassium channel agonists XX-03-64 (least active) and XX-03-56 (most active). The graphs show macroscopic currents recorded from HEK293 cells transfected with GFP-tagged human KCNN3, transcript variant 1 cDNA (Accession # NM_002249), and co-expressed with SK3 in the whole-cell patch clamp configuration at 22-24° C. FIG. 1A (insert) shows the protocol for receptor activation. Currents were activated by 200 msec depolarizing ramps between −120 mV and +30 mV, from a holding potential of −90 mV, and then deactivated by repolarization to the holding potential. FIG. 1A shows whole cell current characteristics as a function of voltage for control (black), XX-03-64-treated (blue), and XX-03-56-treated (red) samples. FIG. 1B shows cellular response over time. Colored bars indicate application of the XX-03-64 and XX-03-56 agonists.

FIG. 2 is a schematic representation of endothelial and smooth muscle cells showing how indirectly or directly hyperpolarizing the plasma membrane potential of VSM may inhibit Ca²⁺ influx, inducing relaxation and offering an effective therapeutic approach for HF.

FIG. 3 is a schematic representation of vascular ion channels. The top part of the schematic is an endothelial cell. Activation of SK3 and IK1 channels hyperpolarizes the endothelial cell membrane potential which indirectly promotes smooth muscle hyperpolarization and relaxation by: (i) increasing the production of nitric oxide (NO) and arachidonic acid metabolites; (ii) activating Na⁺—K⁺ ATPase and K_{IR} channels in underlying VSM; and (iii) myo-endothelial gap junctions. The bottom part of the schematic shows a VSM cell. Elevation of intraluminal pressure activates TRP channels, causing depolarization, activation of Ca²⁺ channels (Ca_{V}), elevation of Ca²⁺ᵢ concentration and vasoconstriction. Vasoconstriction is attenuated by BK currents that are activated by Ca²⁺ sparks from ryanodine receptors (RyR). BK channels are important for the nitric acid (NO), epoxyeicosatrienoic acids (EET) and hydrogen peroxide (H₂O₂) mediated relaxation. BK channels are not required for EDHF-induced relaxation.

FIGS. 4A-4G collectively show that myogenic tone is increased in HF. FIGS. 4A and 4B are Masson trichrome-stained cardiac cross-sections. Blue shows infarcted tissue, whereas red dye areas indicate viable tissue. FIGS. 4C and 4D are graphs of ejection fraction and fractional shortening 6 weeks post-surgery. Mean+s.e.m. ***P≤0.001 *P≤0.01; n=14 sham, n=56 LAD-ligated. FIGS. 4E and 4F show representative changes in internal vessel diameter (upper) in response to changes in intraluminal pressure from a sham mouse and a LAD-ligated mouse with HF. FIG. 4G is a graph of intraluminal pressure vs. myogenic constriction (tone) from mice without and with HF. Mean+s.e.m. No HF: N=6 mice; HF: N=10 mice. * P<0.05, *** P<0.001; repeated measures 2-way ANOVA.

FIG. 5 is a graph showing survival of sham-operated and LAD-ligated WT and BK β1 mice. A log-rank test was performed comparing the empiric (Kaplan-Meier) cumulative distribution functions for the two populations of interest (LAD-ligated WT and LAD-ligated BK β1 null). The null hypothesis, that these two populations have the same cumulative distribution function, can be rejected with a P=0.03. The dashed lines are 95% confidence intervals, computed using Greenwood's formula. N=10 WT sham, 18 WT LAD-ligated, 14 BK β1 null LAD-ligated. Note: Within the first 24 hours post-MI, WT mice have about 10% mortality due to post-operative bleeding, arrhythmias, HF, or ongoing ventilator dependence. These complications were even more common in BK β1 null mice, but to be as conservative, mice that died within the first 24 hours of LAD-ligation were excluded.

FIGS. 6A-6G collectively show that LV function is reduced in LAD-ligated BK β1 mice survivors. FIGS. 6A and 6B show plastic casts of the coronary arterial system of male WT and BK β1 null mice. FIGS. 6C and 6D show 2-D (upper) and M-mode echocardiography (lower) of LAD-ligated WT (FIG. 6C) and LAD-ligated BK β1 null mice (FIG. 6D). LVEDD, LV end-diastolic diameter; LVESD, LV end-systolic diameter. FIG. 6E is a bar graph of LV ejection fraction (EF); mean+s.e.m. P=0.02 by student's t-test; N=7 WT LAD-ligated, 4 BK β1 null LAD-ligated. FIGS. 6F and 6G are representative hematoxylin and eosin stained sections of 3 LAD-ligated WT and 3 BK β1 null mice, 6 weeks after MI. Scale bars=1.0 mm.

FIG. 7 is a graph showing increased VSM depolarization in mesenteric resistance vessels from mice with HF. VSM membrane potential at 40, 80 and 120 mm Hg. N=3-7 mice for each group and pressure. Mean+s.e.m. * P<0.05, ** P<0.01; repeated measures 2-way ANOVA.

FIGS. 8A-8F collectively show graphs presenting RT-qPCR and Affymetrix microarray analysis of mesenteric arteries from HF and sham mice. FIG. 8A is a graph showing vasodilatory K⁺ channels and transporters. FIG. 8B is a graph showing TRP, Orai, and STIM. FIG. 8C is a graph showing voltage-gated Ca²⁺ channels. FIG. 8D is a graph showing intracellular Ca²⁺ release channels. FIG. 8E is a graph showing Ca²⁺-activated Cl⁻ channels. Error bars=(average fold change)×(2^{SEM}−1). Data were normalized using the Robust Multi-Array Average (RMA) (Irizarry et al. 2003), and the differences were analyzed by 1-way ANOVA using NIA Array Analysis software (Bayesian Error Model and 10 degrees of freedom). The statistical significance was determined using the False Discovery Rate (FDR) method (Benjamini et al. 1995). The preliminary data were obtained from five male mice with HF and sham-controls. FIG. 8F is a graph showing mRNA levels from real-time qPCR of BK α, BK β1 and K_{V}1.5, run in duplicate and analyzed using ΔΔCT comparisons, normalized to GAPDH and to sham (set to 1). *P<0.05; N=3 animals. Mean+SD of 3 separate experiments.

FIGS. 9A-9F collectively show decreased BK expression in HF mice. Representative DAB-IHC of transverse sections of third-order mesenteric vessels are shown. BK α and β1 were detected using anti-BK α and anti-BK β1 antibodies and DAB. Scales bars are 50 μm and 20 μm for low and high power images, respectively. FIG. 9A shows BK α null and FIG. 9E shows BK β1 null. Insets are immunoblots of extracts from BK α and β1 null mice showing specificity of the antibodies. The multiple bands represent varying glycosylated β1 subunits (see Wu et al. 2013). FIGS. 9B and 9F are no HF, WT mice; FIGS. 9C and 9G are HF mice; and FIGS. 9D and 9H are graphs showing quantification of DAB-positive staining as a fraction of vessel area. **P<0.01.

FIGS. 10A-10C collectively show that STOCs are decreased in HF mice. FIG. 10A shows representative traces of transient BK currents at −20 mV and 0 mV, recorded from freshly isolated mesenteric artery VSM cells of sham, and LAD-ligated HF mice. *=time-point of traces on right. FIGS. 10B and 10C are graphs showing STOC amplitude and frequency. Mean+s.e.m. No HF (No HF): N=7; HF (HF) N=12. *P<0.05,** P<0.01 by t test.

FIGS. 11A-11B collectively show that the inhibition of BK channels increases myogenic constriction in control mice, but not mice with heart failure. FIG. 11A is a graph showing the effect of paxilline on myogenic tone measured at 120 mm Hg. Constriction induced by paxilline was calculated as the difference between myogenic tone (%) pre- and post-paxilline. Mean+s.e.m. No heart failure (no HF): N=6 vessels; heart failure (HF): N=5 vessels. ** P<0.01, Student's t test. FIG. 11B is a graph showing that myogenic tone is equivalent in paxilline-treated vessels from control mice and mice with heart failure. Mean+s.e.m. No HF: N=6 vessels; HF: N=5 vessels.

FIG. 12 is a graph showing the effect of intraluminal pressure on myogenic tone in mesenteric arteries from BK β1 null mice with and without HF. HF does not significantly increase myogenic constriction in BK β1 null mice. Mean+s.e.m. No HF: N=6; HF: N=8 mice. P>0.05 (not significant) by repeated measures 2-way ANOVA and generalized estimating equations.

FIGS. 13A-13E collectively show confocal images of frozen sections of aorta stained with anti-BK α antibody. In FIGS. 13A, 13B and 13C, BK channel was detected by in situ PLA with anti-mouse PLUS and MINUS PLA probes. Images were obtained using confocal, 60×. Auto-fluorescence of internal elastic lamina is also detected as shown in the negative control (FIG. 13C) in which no primary antibody was used. Scale bars=20 μm. FIGS. 13D and 13E show auto-detection and quantitation of PLA signals using the Duolink image tool. PLA signals are marked with white circles. Images were analyzed using same settings, pixel size 3, signal intensity 450. FIG. 13F is a bar graph of quantification of BK “spots”.

FIGS. 14A-14B collectively show the inhibition of BK currents by paxilline and Ang II in freshly isolated mesenteric VSM cells. Whole cell K⁺ currents at baseline (black trace) and after exposure to 1 μM paxilline (red trace in FIG. 14A) or 2 μM Ang II (blue trace in FIG. 14B). The intrapipette solution contained 9 μM Ca²⁺.

FIGS. 15A-15C collectively show data from BK α-expressing Tg mice. FIG. 15A is a picture of a gel showing PCR of genomic DNA. Mouse #1, 2, 4 and 6 are positive for both BK α (upper panel) and SM22α-rtTA (lower panel). FIG. 15B is a picture of an immunoblot showing anti-BK α antibody against aortic lysates from Tg-negative and four double Tg BK α mice fed doxycycline. Three of four mouse lines show increased BK α expression compared to control. Equal amount of protein, assessed by Bradford, was loaded in each lane. FIG. 15C is a graph showing BK current (nA/pF) at +160 mV. BK currents were significantly increased in BK α mice. Mean+s.e.m. *P<0.05. WT littermate: N=9 cells from 6 mice; double Tg BK α: N=6 cells from 3 mice.

FIGS. 16A-16B collectively show data from BK β1-expressing Tg mice. FIG. 16A shows the results of a PCR of genomic DNA with primers spanning vector and BK β1 subunit (upper panel) and SM22α-rtTA (lower panel). All 3 of these mice are positive for both transgenes. FIG. 16B is an anti-BK β1 immunoblot of aortic lysates from a transgene negative and double transgene positive mouse. Equal amount of protein, assessed by Bradford reagent, was loaded in each lane.

FIG. 17 is a schematic diagram depicting the results of experiments disclosed in Example 14 of the present invention.

FIGS. 18A-18D collectively are graphs showing electrophysiological characterization of SK channels in freshly isolated mesenteric endothelial cells. FIGS. 18A and 18B show reversible NS-309 (5 μM) activation of SK/IK channels. Time course of NS-309 activation at 100 mV is shown in FIG. 18A. FIGS. 18C and 18D show NS-309 and carbachol (2 μM)-induced hyperpolarization of endothelial cell membrane potential.

FIGS. 19A-19C are graphs collectively showing that SKA-31 dilates mesenteric resistance vessels isolated from both control and HF mice. Vessels were mounted onto cannulas and equilibrated at 80 mm Hg. SKA-31 was perfused into cannulated vessels and luminal diameter was monitored continuously. The effect of SKA-31 is presented relative to the maximal dilation of the vessel in a Ca²⁺-free solution. FIG. 19A shows changes in internal vessel diameter of a mesenteric artery isolated from mice with HF. FIG. 19B is a graph of SKA-31 induced dilation relative to maximal dilation determined in Ca²⁺-free solution. Mean+s.e.m, N=4-5 vessels. FIG. 19C is a graph showing that the infusion of 1 μM TRAM-34 and 100 nM apamin blocked SKA-31 induced vasodilation of mesenteric vessels. N=3.

FIG. 20 is a graph showing that SKA-31 dilates BK β1 null mesenteric resistance vessels. The vessels were continuously constricted with 1 μM phenylephrine. SKA-31 was perfused through the cannulas. At the conclusion of the experiment, the vessels were superfused in a Ca²⁺-free solution to determine maximal dilation. Data shown are mean+s.e.m., relative to maximal dilation determined in Ca²⁺-free solution. N=4.

FIG. 21 is a schematic diagram showing the results of experiments disclosed in Example 15 of the present invention.

FIG. 22 depicts data of a wildtype mouse with heart failure (FAC 38%) given an IV bolus of SKA-31 (3 mg/kg) through the left internal jugular vein during simultaneous recording of left ventricular pressure and volume using a Milar conductance catheter inserted through the right carotid artery.

### DETAILED DESCRIPTION OF THE INVENTION

One embodiment of the present invention is a compound having formula (I):

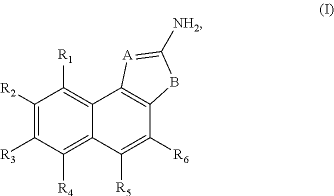

wherein
A and B are independently selected from the group consisting of S or N;
R₁, R₂, R₃, R₄, and R₆ are independently selected from the group consisting of H, halogen, alkyl, ester, ether, thioether, aryl, heteroaryl, CN, NO₂, and amine;
R₅, is selected from the group consisting of H, ester, thioether, aryl, heteroaryl, NO₂, and amine;
wherein each alkyl is optionally substituted with a group consisting of halide, ether, and combinations thereof;
wherein each aryl and each heteroaryl are optionally substituted with a group consisting of halide, ether, C₁₋₄alkyl, and combinations thereof; and
wherein each amine is optionally substituted with a group selected from halide, C₁₋₄alkyl, and combinations thereof,
   and crystalline forms, hydrates, or pharmaceutically acceptable salts thereof,
   with the provisio that the compound is not

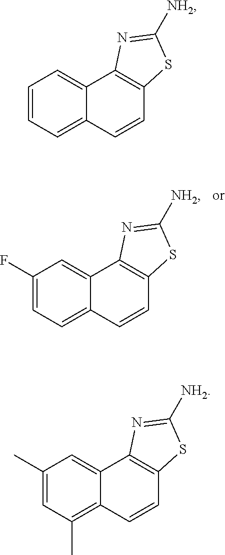 [SMILES: Nc1nc2c(ccc3ccccc32)s1 ; Nc1nc2c(ccc3ccc(F)cc32)s1 ; Cc1cc(C)c2ccc3sc(N)nc3c2c1]

The term “aryl” as used herein includes substituted or unsubstituted single-ring aromatic groups in which each atom of the ring is carbon. Preferably the ring is a 3- to 8-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “substituted” means moieties having substituents replacing a hydrogen on one or more carbons of the backbone. It will be understood that “substitution” or “substituted with” includes the implicit proviso that such substitution is in accordance with the permitted valence of the substituted atom and the substituent, and that the substitution results in a stable compound, e.g., which does not spontaneously undergo transformation such as by rearrangement, cyclization, elimination, etc. The permissible substituents can be one or more and the same or different for appropriate organic compounds.

As used herein, a “halide” means a halogen atom such as fluorine, chlorine, bromine, iodine, or astatine.

As used herein, an “aromatic ring” is an aryl or a heteroaryl. The term “heteroaryl” includes substituted or unsubstituted aromatic single ring structures, preferably 3- to 8-membered rings, more preferably 5- to 7-membered rings, even more preferably 5- to 6-membered rings, whose ring structures include at least one heteroatom, preferably one to four heteroatoms, more preferably one or two heteroatoms. The term “heteroaryl” also includes polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur; more preferably, nitrogen and oxygen or nitrogen and sulfur.

The terms “amine” and “amino” are art-recognized and refer to both unsubstituted and substituted amines and salts thereof, e.g., a moiety that can be represented by

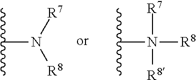

wherein R⁷, R⁸, and R^{8′} each independently represent a hydrogen or a hydrocarbyl group, or R⁷ and R⁸ taken together with the N atom to which they are attached complete a heterocycle having from 4 to 8 atoms in the ring structure. The term “primary” amine means only one of R⁷ and R⁸ or one of R⁷, R⁸, and R^{8′} is a hydrocarbyl group. Secondary amines have two hydrocarbyl groups bound to N. In tertiary amines, all three groups, R⁷, R⁸, and R^{8′}, are replaced by hydrocarbyl groups.

As used herein, the term “heterocycle” means substituted or unsubstituted non aromatic ring structures. Preferably the heterocycle comprises 3 to 8 membered rings, and at least one heteroatom, preferably one to four heteroatoms, more preferably one or two heteroatoms. Such heterocycles may include at least one ring nitrogen. The term “heterocycle” also includes polycyclic ring systems having two or more cyclic rings in which two or more carbons are common to two adjoining rings wherein at least one of the rings is heterocyclic, e.g., the other cyclic ring(s) can be cycloalkyls, cycloalkenyls, cycloalkynyls, aryls, heteroaryls, and/or heterocyclyls. Heterocycle groups of the present invention include, for example, piperidine, piperazine, pyrrolidine, morpholine, lactones, lactams, and the like.

The term “alkyl” means the radical of saturated aliphatic groups that does not have a ring structure, including straight-chain alkyl groups, and branched-chain alkyl groups. In certain embodiments, a straight chain or branched chain alkyl has 6 or fewer carbon atoms such as 4 or fewer carbon atoms in its backbone (e.g., C₁-C₆ for straight chains, C₃-C₆ for branched chains).

The term “ether”, as used herein, means a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The term “thioether”, as used herein, means a hydrocarbyl group linked through an sulfur to another hydrocarbyl group. Accordingly, an ether substituent of a hydrocarbyl group may be hydrocarbyl-S—. Thioethers may be either symmetrical or unsymmetrical. Examples of thioethers include, but are not limited to, heterocycle-S-heterocycle and aryl-S-heterocycle. Thioethers include groups, which may be represented by the general formula alkyl-S-alkyl.

The term “aliphatic”, as used herein, means a group composed of carbon and hydrogen atoms that does not contain aromatic rings. Accordingly, aliphatic groups include alkyl, alkenyl, alkynyl, and carbocyclyl groups. A preferred C₁₋₄ aliphatic is a vinyl moiety.

The term “alkenyl”, as used herein, means an aliphatic group containing at least one double bond.

The term “alkynyl”, as used herein, means an aliphatic group containing at least one triple bond.

In the present invention, the term “crystalline form” means the crystal structure of a compound. A compound may exist in one or more crystalline forms, which may have different structural, physical, pharmacological, or chemical characteristics. Different crystalline forms may be obtained using variations in nucleation, growth kinetics, agglomeration, and breakage. Nucleation results when the phase-transition energy barrier is overcome, thereby allowing a particle to form from a supersaturated solution. Crystal growth is the enlargement of crystal particles caused by deposition of the chemical compound on an existing surface of the crystal. The relative rate of nucleation and growth determine the size distribution of the crystals that are formed. The thermodynamic driving force for both nucleation and growth is supersaturation, which is defined as the deviation from thermodynamic equilibrium. Agglomeration is the formation of larger particles through two or more particles (e.g., crystals) sticking together and forming a larger crystalline structure.

The term “hydrates”, as used herein, means a solid or a semi-solid form of a chemical compound containing water in a molecular complex. The water is generally in a stoichiometric amount with respect to the chemical compound.

As used herein, “pharmaceutically acceptable salts” refer to derivatives of the compounds disclosed herein wherein the compounds are modified by making acid or base salts thereof. Examples of pharmaceutically acceptable salts include, but are not limited to, mineral or organic acid salts of basic residues such as amines; alkali or organic salts of acidic residues such as carboxylic acids; and the like. For example, such salts include salts from ammonia, L-arginine, betaine, benethamine, benzathine, calcium hydroxide, choline, deanol, diethanolamine (2,2′-iminobis(ethanol)), diethylamine, 2-(diethylamino)-ethanol, 2-aminoethanol, ethylenediamine, N-ethyl-glucamine, hydrabamine, 1H-imidazole, lysine, magnesium hydroxide, 4-(2-hydroxyethyl)-morpholine, piperazine, potassium hydroxide, 1-(2-hydroxy-ethyl)-pyrrolidine, sodium hydroxide, triethanolamine (2,2′,2″-nitrilotris(ethanol)), tromethamine, zinc hydroxide, acetic acid, 2.2-dichloro-acetic acid, adipic acid, alginic acid, ascorbic acid, L-aspartic acid, benzenesulfonic acid, benzoic acid, 2,5-dihydroxybenzoic acid, 4-acetamido-benzoic acid, (+)-camphoric acid, (+)-camphor-10-sulfonic acid, carbonic acid, cinnamic acid, citric acid, cyclamic acid, decanoic acid, dodecylsulfuric acid, ethane-1,2-disulfonic acid, ethanesulfonic acid, 2-hydroxy-ethanesulfonic acid, ethylenediamonotetraacetic acid, formic acid, fumaric acid, galacaric acid, gentisic acid, D-glucoheptonic acid, D-gluconic acid, D-glucuronic acid, glutamic acid, glutantic acid, glutaric acid, 2-oxo-glutaric acid, glycero-phosphoric acid, glycine, glycolic acid, hexanoic acid, hippuric acid, hydrobromic acid, hydrochloric acid isobutyric acid, DL-lactic acid, lactobionic acid, lauric acid, lysine, maleic acid, (−)-L-malic acid, malonic acid, DL-mandelic acid, methanesulfonic acid, galactaric acid, naphthalene-1,5-disulfonic acid, naphthalene-2-sulfonic acid, 1-hydroxy-2-naphthoic acid, nicotinic acid, nitric acid, octanoic acid, oleic acid, orotic acid, oxalic acid, palmitic acid, pamoic acid (embonic acid), phosphoric acid, propionic acid, (−)-L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid and undecylenic acid. Further pharmaceutically acceptable salts can be formed with cations from metals like aluminum, calcium, lithium, magnesium, potassium, sodium, zinc and the like. (also see Pharmaceutical salts, Berge, S. M. et al., J. Pharm. Sci., (1977), 66, 1-19).

The pharmaceutically acceptable salts of the present invention can be synthesized from a compound disclosed herein which contains a basic or acidic moiety by conventional chemical methods. Generally, such salts can be prepared by reacting the free acid or base forms of these compounds with a sufficient amount of the appropriate base or acid in water or in an organic diluent like ether, ethyl acetate, ethanol, isopropanol, or acetonitrile, or a mixture thereof.

Another embodiment of the present invention is a compound having the formula (II)

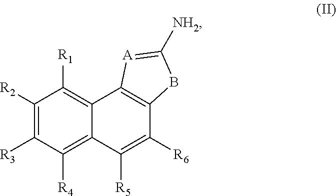

wherein
A and B are independently selected from the group consisting of S or N;
R₁, R₂, R₃, R₄, and R₆ are independently selected from the group consisting of H, halogen, C₁₋₆alkyl, —X—C₁₋₆alkyl, CN, —NO₂, —C(O)—R₇ and —N(R₇)(R₈);
R₅, is selected from the group consisting of H, —NO₂, —C(O)—R₇ and N(R₇)(R₈);
wherein X is independently selected from the group consisting of S or O;
wherein Y is selected from the group consisting of no atom, S or O; and
R₇ and R₈ are independently selected from the group consisting of H, halogen, C₁₋₆alkyl, and CN,
   and crystalline forms, hydrates, or pharmaceutically acceptable salts thereof.

The term “C_{x-y}” when used in conjunction with a chemical moiety, such as, alkyl, alkenyl, or alkoxy is meant to include groups that contain from x to y carbons in the chain. For example, the term “C_{x-y}alkyl” means substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

Another embodiment of the present invention is a compound selected from the group consisting of

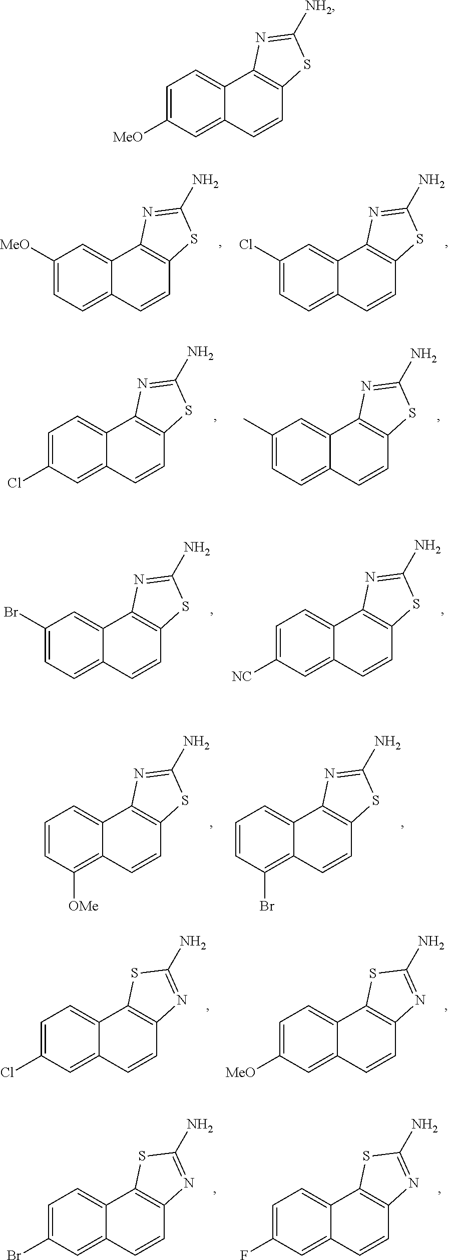

and crystalline forms, hydrates, or pharmaceutically acceptable salts thereof.

Another embodiment of the present invention is a compound having the structure:

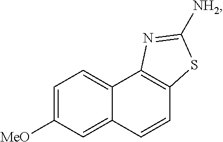 [SMILES: COc1ccc2c(ccc3sc(N)nc32)c1]

or a crystalline form, hydrate, or pharmaceutically acceptable salt thereof.

Another embodiment of the present invention is a compound having the structure:

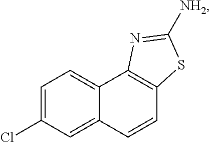 [SMILES: Nc1nc2c(ccc3cc(Cl)ccc32)s1]

or a crystalline form, hydrate, or pharmaceutically acceptable salt thereof.

Another embodiment of the present invention is a pharmaceutical composition comprising a pharmaceutically acceptable carrier and a compound of the present invention.

The compounds or compositions, including pharmaceutical compositions of the present invention may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, compounds or compositions, including pharmaceutical compositions of the present invention may be administered in conjunction with other treatments. Compounds or compositions, including pharmaceutical compositions of the present invention may be encapsulated or otherwise protected against gastric or other secretions, if desired.

The compositions, including pharmaceutical compositions of the invention may comprise one or more active ingredients in admixture with one or more pharmaceutically-acceptable diluents or carriers and, optionally, one or more other compounds, drugs, ingredients and/or materials. Regardless of the route of administration selected, the agents/compounds of the present invention are formulated into pharmaceutically-acceptable dosage forms by conventional methods known to those of skill in the art. See, e.g., Remington, The Science and Practice of Pharmacy (21^{st} Edition, Lippincott Williams and Wilkins, Philadelphia, Pa.).

Pharmaceutically acceptable diluents or carriers are well known in the art (see, e.g., Remington, The Science and Practice of Pharmacy (21^{st} Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and triglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, salicylate, etc. Each pharmaceutically acceptable diluent or carrier used in a pharmaceutical composition of the invention must be “acceptable” in the sense of being compatible with the other ingredients of the formulation and not injurious to the subject. Diluents or carriers suitable for a selected dosage form and intended route of administration are well known in the art, and acceptable diluents or carriers for a chosen dosage form and method of administration can be determined using ordinary skill in the art.

The compositions, including pharmaceutical compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

The compositions, including pharmaceutical compositions of the present invention suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable diluents or carriers and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

The compositions, including pharmaceutical compositions of the present invention for rectal or vaginal administration may be presented as a suppository, which may be prepared by mixing one or more active ingredient(s) with one or more suitable nonirritating diluents or carriers which are solid at room temperature, but liquid at body temperature and, therefore, will melt in the rectum or vaginal cavity and release the active compound. The pharmaceutical compositions of the present invention which are suitable for vaginal administration also include pessaries, tampons, creams, gels, pastes, foams or spray formulations containing such pharmaceutically-acceptable diluents or carriers as are known in the art to be appropriate.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable diluent or carrier. The ointments, pastes, creams and gels may contain excipients. Powders and sprays may contain excipients and propellants.

The compositions, including pharmaceutical compositions of the present invention suitable for parenteral administrations may comprise one or more agent(s)/compound(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These pharmaceutical compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally-administered agent/drug may be accomplished by dissolving or suspending the active agent/drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter. Any formulation of the invention may be presented in unit-dose or multi-dose sealed containers, for example, ampules and vials, and may be stored in a lyophilized condition requiring only the addition of the sterile liquid diluent or carrier, for example water for injection, immediately prior to use. Extemporaneous injection solutions and suspensions may be prepared from sterile powders, granules and tablets of the type described above.

In some aspects of this embodiment, the pharmaceutical composition comprises one or more additional active agents. In a preferred aspect of this embodiment, the additional active agents are selected from the group consisting of nitrates, angiotensin-converting enzyme (ACE) inhibitors, angiotensin receptor blockers, beta-adrenergic blockers, and aldosterone receptor antagonists.

As used herein, a “blocker”, “antagonist” or “inhibitor” means a substance which can reduce the activity or the expression of the target protein. As used herein, an “agonist” means a substance which can activate a receptor.

In one aspect of this embodiment, the additional active agent is a nitrate. Nitrates are venodialators that are thought to reduce the workload of the heart. Nitrates include, without limitation, nitroglycerin, isorbide mononitrate, isosorbide dinitrate, pentaerythrityl tetranitrate, sodium nitroprusside, molsidomine, and SIN-1.

In another aspect of this embodiment, the additional active agent is an ACE inhibitor. ACE inhibitors block the conversion of angiotensin I (AI) to angiotensin II (AII). ACE inhibitors include, without limitation, alacepril, alatriopril, altiopril calcium, ancovenin, benazepril, benazepril hydrochloride, benazeprilat, benzoylcaptopril, captopril, captopril-cysteine, captopril-glutathione, ceranapril, ceranopril, ceronapril, cilazapril, cilazaprilat, delapril, delapril-diacid, enalapril, enalaprilat, enapril, epicaptopril, foroxymithine, fosfenopril, fosenopril, fosenopril sodium, fosinopril, fosinopril sodium, fosinoprilat, fosinoprilic acid, glycopril, hemorphin-4, idrapril, imidapril, indolapril, indolaprilat, libenzapril, lisinopril, lyciumin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spiropril hydrochloride, temocapril, temocapril hydrochloride, teprotide, trandolapril, trandolaprilat, utibapril, zabicipril, zabiciprilat, zofenopril, zofenoprilat, casokinins, lactokinins and lactotripeptides such as Val-Pro-Pro and Ile-Pro-Pro.

In another aspect of this embodiment, the additional active agent is an angiotensin receptor blocker. Angiotensin receptors are a class of G protein-coupled receptors with angiotensin II as their ligands. There are at least four subtypes of angiotensin receptors, type 1, type 2, type 3, and type 4. Angiotensin receptor blockers include, without limitation, candesartan, candesartan cilexetil, losartan, valsartan, irbesartan, tasosartan, telmisartan, eprosartan, L158,809, saralasin and olmesartan.

In another aspect of this embodiment, the additional active agent is a beta-adrenergic blocker. Beta-adrenergic blocker are beta-adrenoreceptor antagonists which include, without limitation, acebutolol, atenolol, betaxolol, bevantolol, bisoprolol, celiprolol, cetamolol, epanolol, esmolol, levobetaxolol, practolol, propranolol, bucindolol, carteolol, carvedilol, nadolol, oxyprenolol, penbutolol, pindolol, sotalol, timolol, metoprolol, nebivolol, butaxamine, IC-118,551 and SR59230A.

In a further aspect of this embodiment, the additional active agent is a aldosterone receptor antagonist. Aldosterone receptor antagonists are diuretics that help the body get rid of extra water. Aldosterone receptor antagonists include, without limitation, spironolactone, eplerenone, canrenone, propenone and mexrenone.

In the present invention, the additional active agents may be used as a single agent together with the compounds and compositions, including pharmaceutical compositions of the present invention. The additional active agents may also be used with one or more additional active agents together with the compounds and compositions, including pharmaceutical compositions.

Another embodiment of the present invention is a method for treating or ameliorating the effects of a condition in a subject in need thereof comprising administering to the subject an effective amount of a compound of the present invention or a pharmaceutical composition of the present invention. In a preferred aspect of this embodiment, the condition is high blood pressure or diabetes. In a more preferred aspect of this embodiment, the condition is heart failure syndrome.

In the present invention, an “effective amount” or a “therapeutically effective amount” of a compound or composition, including pharmaceutical compositions disclosed herein is an amount of such compound or composition that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of a compound or composition, including pharmaceutical compositions according to the invention will be that amount of the compound or composition which is the lowest dose effective to produce the desired effect. The effective dose of a compound or composition, including pharmaceutical compositions of the present invention may be administered as two, three, four, five, six or more sub-doses, administered separately at appropriate intervals throughout the day.

A suitable, non-limiting example of a dosage of any of the compounds or compositions, including pharmaceutical compositions disclosed herein is from about 1 mg/kg to about 2400 mg/kg per day, such as from about 1 mg/kg to about 1200 mg/kg per day, 75 mg/kg per day to about 300 mg/kg per day, including from about 1 mg/kg to about 100 mg/kg per day. Other representative dosages of such agents include about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, 1000 mg/kg, 1100 mg/kg, 1200 mg/kg, 1300 mg/kg, 1400 mg/kg, 1500 mg/kg, 1600 mg/kg, 1700 mg/kg, 1800 mg/kg, 1900 mg/kg, 2000 mg/kg, 2100 mg/kg, 2200 mg/kg, and 2300 mg/kg per day. The effective dose of compounds or compositions, including pharmaceutical compositions disclosed herein, may be administered as two, three, four, five, six or more sub-doses, administered separately at appropriate intervals throughout the day.

As used herein, the terms “ameliorate”, “ameliorating” and grammatical variations thereof mean to decrease the severity of the symptoms of a disease in a subject.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions, including pharmaceutical compositions of the present invention may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population, may fail to respond or respond inadequately to treatment.

As used herein, a “subject” is a mammal, preferably, a human. In addition to humans, categories of mammals within the scope of the present invention include, for example, primates, farm animals, domestic animals, laboratory animals, etc. Some examples of agricultural animals include cows, pigs, horses, goats, etc. Some examples of domestic animals include dogs, cats, etc. Some examples of laboratory animals include primates, rats, mice, rabbits, guinea pigs, etc.

Another embodiment of the present invention is a method for treating or ameliorating the effects of heart failure syndrome (HF) in a subject in need thereof comprising administering to the subject an effective amount of a pharmaceutical composition of the invention. In this embodiment, the subject is a mammal as described above, preferably a human.

Another embodiment of the present invention is a kit for treating or ameliorating the effects of a condition in a subject in need thereof. In this embodiment, the kit comprises an effective amount of a compound of the present invention or a pharmaceutical composition of the present invention, packaged together with instructions for its use. In a preferred aspect of this embodiment, the condition is high blood pressure or diabetes. In a more preferred aspect of this embodiment, the condition is heart failure syndrome.

The kits of the present invention may also include suitable storage containers, e.g., ampules, vials, tubes, etc., for the compounds and compositions, including pharmaceutical compositions of the present invention and other active agents and/or reagents, e.g., buffers, balanced salt solutions, etc., for use in administering the compounds and compositions to subjects. The compounds and compositions, including pharmaceutical compositions of the present invention may be present in the kits in any convenient form, such as, e.g., in a solution or in a powder form. The kits may further include a packaging container, optionally having one or more partitions for housing the compounds and pharmaceutical compositions and other optional reagents.

Another embodiment of the present invention is a kit for treating or ameliorating the effects of heart failure syndrome (HF) in a subject in need thereof. In this embodiment, the kit comprises an effective amount of a pharmaceutical composition of the present invention, packaged together with instructions for its use.

Another embodiment of the present invention is a composition comprising a compound of the present invention. In one aspect of this embodiment, the composition is a research reagent. As used herein, a “research reagent” is any compound or composition used in the execution of investigational activities.

The terminology used herein is for the purpose of describing particular embodiments only and is not intended to be limiting. As used in the specification and the appended claims, the singular forms “a,” “an,” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

### EXAMPLES

The following examples are provided to further illustrate certain aspects of the present invention. These examples are illustrative only and are not intended to limit the scope of the invention in any way.

### Example 1

### Synthesis Reactions of KCa Potassium Channel Activators; General Synthesis Procedure.

As shown below, the substituted tetralone compounds (0.5 mmol), thiourea (0.5 mmol), PTSA (2.5 mmol) and iodine (0.15 mmol) were mixed in a round bottom flask. DMSO (2 mL) was added and the reaction mixture was stirred under oxygen at 75° C. for 24 hours. The reaction mixture was cooled down and diluted with ethyl acetate. The organic phase was washed with aqueous sodium bicarbonate, water and brine. The resulting organic phase was dried over sodium sulfate and concentrated. The crude product was purified with preparative TLC with ethyl acetate and dichloromethane (ratio 1:3) to provide the product. Compound identities were confirmed by ¹HNMR.

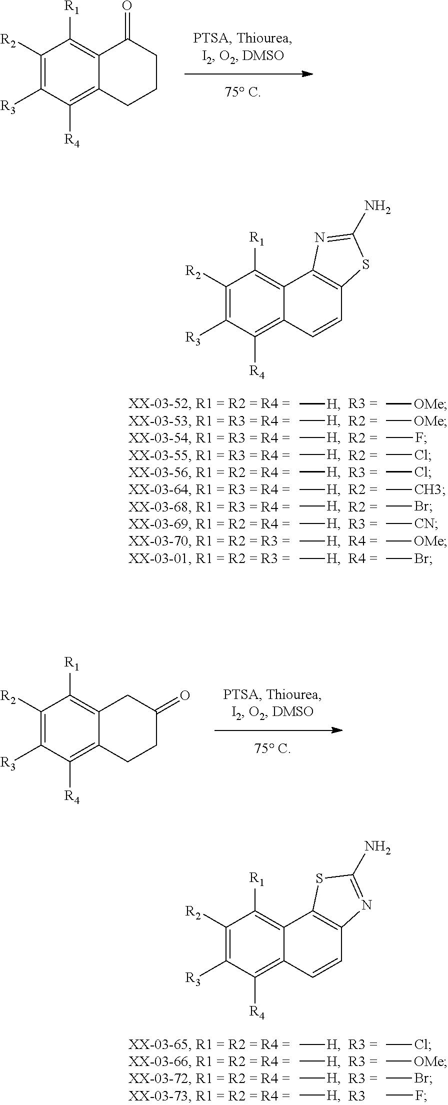

XX-03-52, ¹HNMR (400 MHz, CDCl₃) δ 8.37 (d, J=8.8 Hz, 1H), 7.61 (d, J=8.8 Hz, 1H), 7.50 (d, J=8.4 Hz, 1H), 7.21 (d, J=9.6 Hz, 1H), 7.19 (s, 1H), 5.55 (br s, 2H), 3.93 (s, 3H).

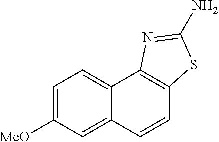 [SMILES: COc1ccc2c(ccc3sc(N)nc32)c1]

XX-03-53, ¹HNMR (400 MHz, CDCl₃) δ 7.80 (d, J=2.8 Hz, 1H), 7.75 (d, J=9.2 Hz, 1H), 7.53 (d, J=8.8 Hz, 1H), 7.50 (d, J=8.4 Hz, 1H), 7.14 (d, J=8.8 Hz, 1H), 7.13 (d, J=8.8 Hz, 1H), 5.70 (br s, 2H), 3.97 (s, 3H); ¹³CNMR (100 MHz, CDCl₃) δ 166.1, 158.1, 146.7, 129.7, 127.9, 127.4, 126.9, 122.5, 118.0, 116.2, 102.1, 55.5.

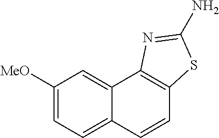 [SMILES: COc1ccc2ccc3sc(N)nc3c2c1]

XX-03-54, ¹HNMR (400 MHz, CDCl₃) δ 8.09 (dd, J=2.4, 10.0 Hz, 1H), 7.85 (dd, J=5.6, 9.2 Hz, 1H), 7.62 (d, J=8.8 Hz, 1H), 7.59 (d, J=9.2 Hz, 1H), 7.25 (dt, J=2.8, 8.6 Hz, 1H), 5.47 (br s, 2H).

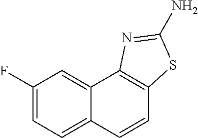 [SMILES: Nc1nc2c(ccc3ccc(F)cc32)s1]

XX-03-55, ¹HNMR (400 MHz, CDCl₃) δ 8.48 (d, J=2.0 Hz, 1H), 7.79 (d, J=8.8 Hz, 1H), 7.66 (d, J=8.8 Hz, 1H), 7.57 (d, J=8.8 Hz, 1H), 7.42 (dd, J=2.0, 8.8 Hz, 1H), 5.38 (br s, 2H).

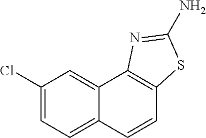 [SMILES: Nc1nc2c(ccc3ccc(Cl)cc32)s1]

XX-03-56, ¹HNMR (400 MHz, CDCl₃) δ 8.43 (d, J=9.2 Hz, 1H), 7.85 (s, 1H), 7.69 (d, J=8.4 Hz, 1H), 7.52 (d, J=8.4 Hz, 1H), 7.49 (d, J=9.2 Hz, 1H), 5.36 (br s, 2H).

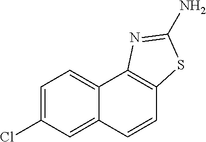 [SMILES: Nc1nc2c(ccc3cc(Cl)ccc32)s1]

XX-03-68, ¹HNMR (400 MHz, CDCl₃) δ 8.65 (d, J=1.2 Hz, 1H), 7.73 (d, J=8.8 Hz, 1H), 7.67 (d, J=8.8 Hz, 1H), 7.56 (d, J=8.8 Hz, 1H), 7.55 (d, J=8.4 Hz, 1H), 5.37 (br s, 2H).

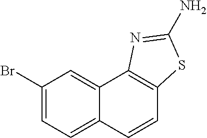 [SMILES: Nc1nc2c(ccc3ccc(Br)cc32)s1]

XX-03-69, ¹HNMR (400 MHz, Acetone-d₆) δ 8.58 (d, J=8.4 Hz, 1H), 8.45 (s, 1H), 7.97 (d, J=9.2 Hz, 1H), 7.74 (d, J=9.2 Hz, 1H), 7.74 (d, J=9.2 Hz, 1H), 7.18 (br s, 2H).

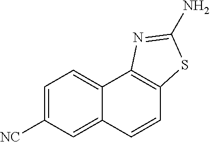 [SMILES: N#Cc1ccc2c(ccc3sc(N)nc32)c1]

XX-03-70, ¹HNMR (400 MHz, Acetone-d₆) δ 8.05 (d, J=8.0 Hz, 1H), 7.93 (d, J=9.2 Hz, 1H), 7.73 (d, J=8.8 Hz, 1H), 7.43 (t, J=8.0 Hz, 1H), 7.06 (br s, 2H), 6.92 (d, J=7.2 Hz, 1H), 4.00 (s, 3H); ¹³CNMR (100 MHz, Acetone-d₆) δ 167.2, 155.6, 148.2, 127.7, 126.7, 125.8, 123.9, 117.9, 116.2, 115.2, 103.6, 55.0.

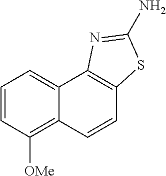 [SMILES: COc1cccc2c1ccc1sc(N)nc12]

XX-03-71, ¹HNMR (400 MHz, Acetone-d₆) δ 8.51 (d, J=8.8 Hz, 1H), 7.92 (d, J=9.2 Hz, 1H), 7.89 (d, J=8.8 Hz, 1H), 7.80 (d, J=7.2 Hz, 1H), 7.42 (d, J=8.0 Hz, 1H), 7.10 (br s, 2H); ¹³CNMR (100 MHz, Acetone-d₆) δ 168.1, 148.7, 130.4, 129.3, 127.7, 127.1, 126.0, 124.1, 122.2, 120.6, 119.7.

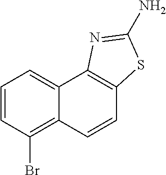 [SMILES: Nc1nc2c(ccc3c(Br)cccc32)s1]

XX-03-72, ¹HNMR (400 MHz, Acetone-d₆) δ 8.14 (d, J=1.6 Hz, 1H), 7.76 (d, J=8.4 Hz, 1H), 7.69 (d, J=8.4 Hz, 1H), 7.66 (d, J=9.2 Hz, 1H), 7.61 (dd, J=1.6, 9.0 Hz, 1H), 7.00 (br s, 2H).

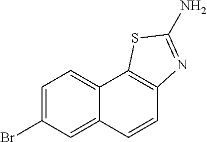 [SMILES: Nc1nc2ccc3cc(Br)ccc3c2s1]

XX-03-73, ¹HNMR (400 MHz, Acetone-d₆) δ 7.79 (dd, J=5.6, 8.4 Hz, 1H), 7.77 (d, J=8.4 Hz, 1H), 7.65 (d, J=8.4 Hz, 1H), 7.65 (d, J=8.4 Hz, 1H), 7.37 (dt, J=2.4, 8.8 Hz, 1H), 6.91 (br s, 2H).

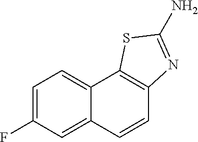 [SMILES: Nc1nc2ccc3cc(F)ccc3c2s1]

### Example 2

### Analysis of KCa Potassium Channel Activator Activity.

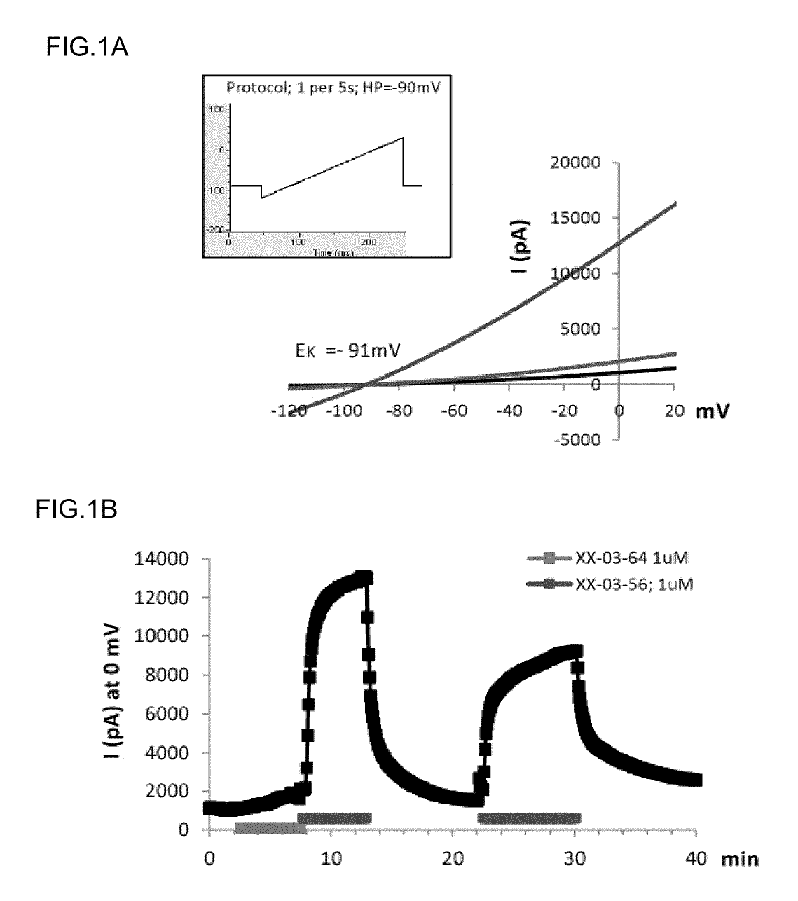

Macroscopic currents were recorded from HEK293 cells transfected with GFP-tagged human KCNN3, transcript variant 1 cDNA (Accession # NM_002249) in the whole-cell patch clamp configuration at 22-24° C. Currents were activated by 200 msec depolarizing ramps between −120 mV and +30 mV, from a holding potential of −90 mV, and then deactivated by repolarization to the holding potential. Voltage ramps were applied every 5 seconds. The bath solution was 140 mM NaCl, 4 mM KCl, 0.1 mM CaCl₂, 3 mM Mg Cl₂, 10 mM HEPES (pH 7.4) and the pipette solution was 150 mM KCl, 5 mM TES (pH 7.2), 2 mM HEDTA, and 1 μM free Ca²⁺. The free Ca²⁺ concentration was calculated using the Max Chelator program and confirmed using a Ca²⁺ electrode (Orion). Agonists were superfused in the bath solution. Results are shown in FIGS. 1A-1B and Table 1 below.

TABLE 1

| Compound | Compound | | | | Activity | | |
|---|---|---|---|---|---|---|---|
| ID | Structure | M.W. | tPSA | CLogP | 0.5 μM | 1 μM | Note |
| XX-03-58 | 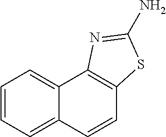 [SMILES: Nc1nc2c(ccc3ccccc32)s1] | 200.26 | 38.38 | 3.00 | + | + | Reference Compound, (Mal Pharmacel, 75, 281,2009) SKA-31, EC50 = 0.26 μM 3rd most active |
| XX-03-52 | 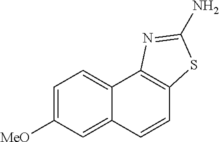 [SMILES: COc1ccc2c(ccc3sc(N)nc32)c1] | 230.29 | 47.61 | 3.04 | + | + | 2nd most active |
| XX-03-53 | 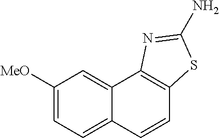 [SMILES: COc1ccc2ccc3sc(N)nc3c2c1] | 230.29 | 47.61 | 3.04 | | + | |
| XX-03-54 | 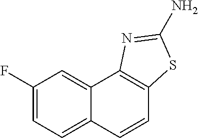 [SMILES: Nc1nc2c(ccc3ccc(F)cc32)s1] | 218.25 | 38.38 | 3.15 | | + | |
| XX-03-55 | 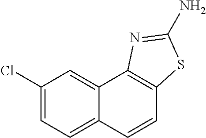 [SMILES: Nc1nc2c(ccc3ccc(Cl)cc32)s1] | 234.70 | 38.38 | 3.72 | | + | |
| XX-03-56 | 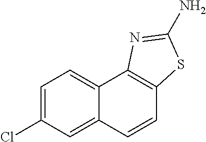 [SMILES: Nc1nc2c(ccc3cc(Cl)ccc32)s1] | 234.70 | 38.38 | 3.72 | + | + | Most active |
| XX-03-64 | 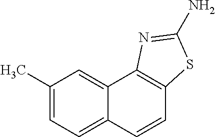 | 214.29 | 38.38 | 3.50 | | o | |
| XX-03-65 | 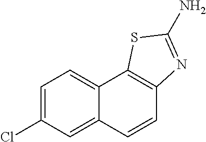 [SMILES: Nc1nc2ccc3cc(Cl)ccc3c2s1] | 234.70 | 38.38 | 3.72 | | | |
| XX-03-66 | 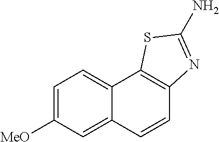 [SMILES: COc1ccc2c(ccc3nc(N)sc32)c1] | 230.29 | 47.61 | 3.04 | | | |
| XX-03-68 | 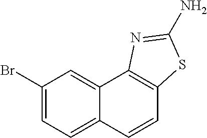 [SMILES: Nc1nc2c(ccc3ccc(Br)cc32)s1] | 279.16 | 38.38 | 3.87 | | | |
| XX-03-69 | 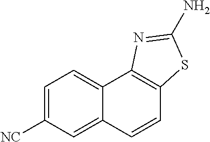 [SMILES: N#Cc1ccc2c(ccc3sc(N)nc32)c1] | 225.27 | 62.17 | 2.46 | | | |
| XX-03-70 | 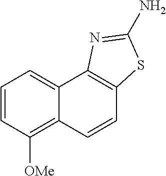 [SMILES: COc1cccc2c1ccc1sc(N)nc12] | 230.29 | 47.61 | 3.04 | | | |

### Example 3

### Myogenic Tone: A Read-Out of the Intrinsic Contractility of Small Resistance Vessels.

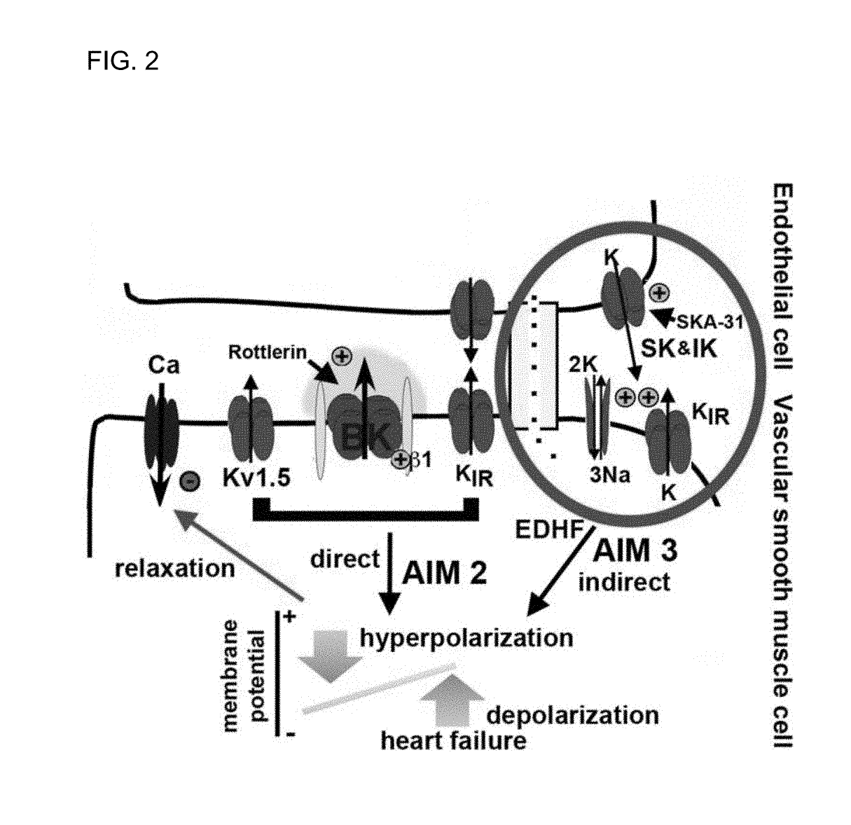

Small arteries constrict in response to increases in luminal pressure, and dilate with decreased pressure, a process referred to as myogenic tone (Bayliss 1902). Arteriolar myogenic tone is an intrinsic property of arterial smooth muscle and occurs independently of systemic factors such as nervous and hormonal inputs. Importantly, myogenic tone underlies the local autoregulation of microvascular blood flow and sets the vascular diameter upon which vasodilators and vasoconstrictors act (Johnson 1986, Hill et al. 2009). Its measurement ex vivo reflects the intrinsic contractility of the small vessels that are fundamental for setting the systemic vascular resistance. Abnormalities in myogenic tone are linked to hypertension, diabetes, stroke and vasospasm (see FIG. 2) (Baek et al. 2011).

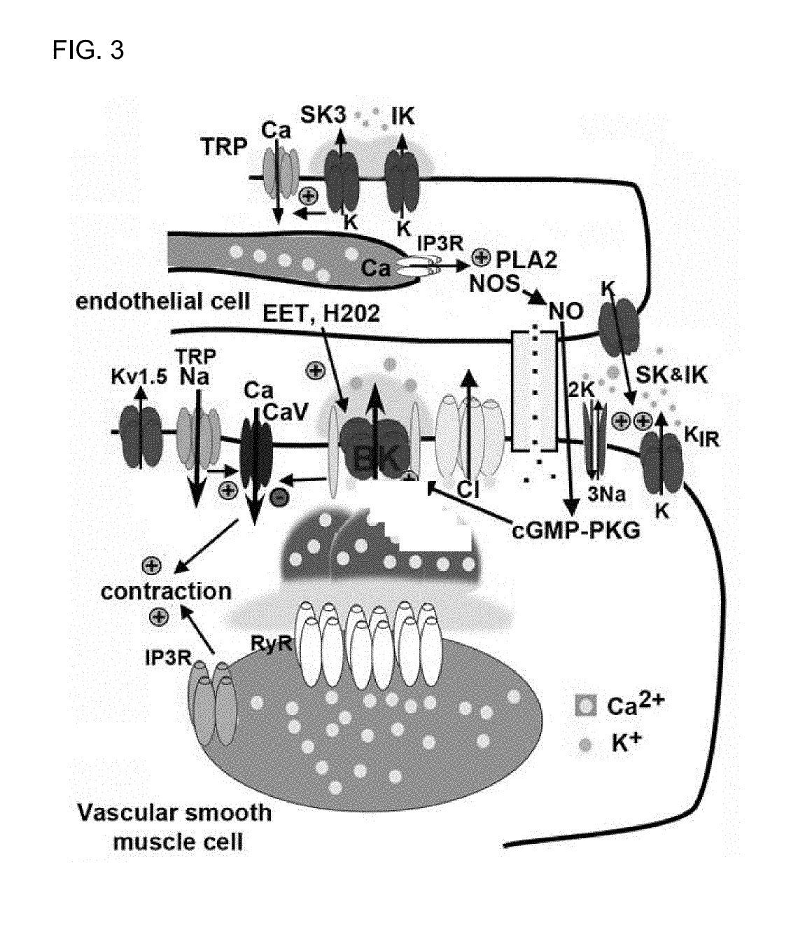

Membrane potential, Ca²⁺ influx and the cytosolic Ca²⁺ concentration within VSM cells are key regulators of myogenic tone and vascular contractility (Hill et al. 2006). Myogenic tone requires pressure and stretch activation of transient receptor potential (TRP) channels, leading to VSM cell depolarization, opening of voltage-gated Ca²⁺ channels, and Ca²⁺ influx, which causes myosin light chain (MLC) phosphorylation and contraction (Earley et al. 2010, Fernandez-Tenorio et al. 2011) (see, e.g., FIG. 3).

### Myogenic Tone is Increased in HF.

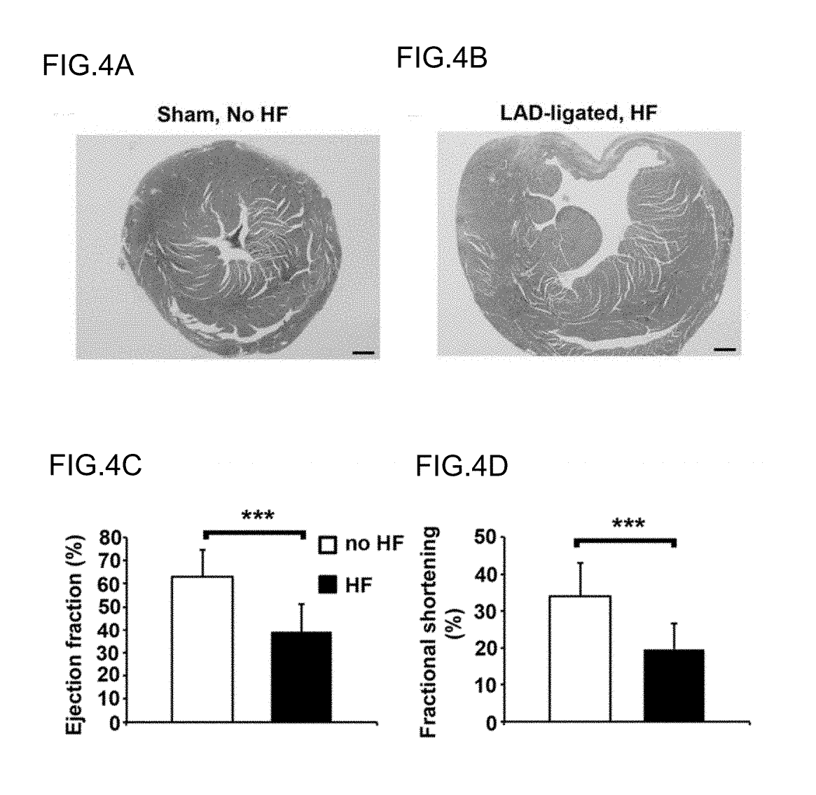

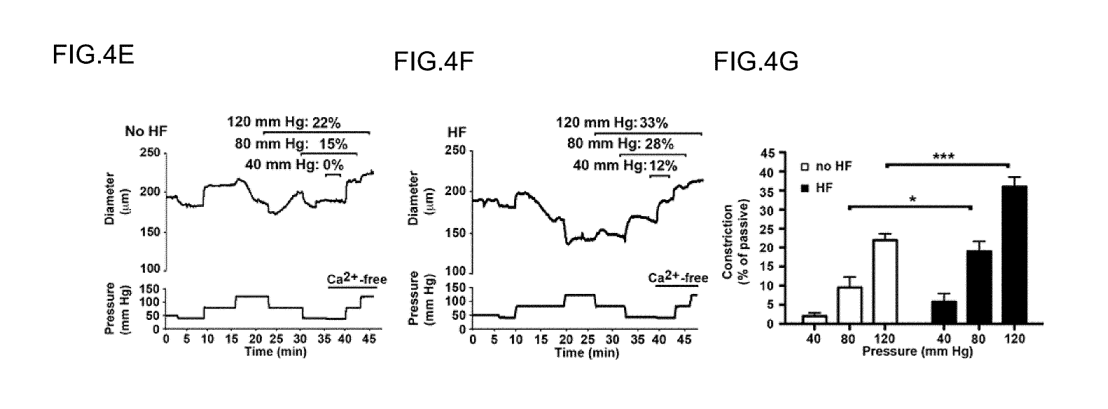

Male C57BL/6 mice underwent left anterior descending (LAD) artery ligation to cause a large MI (see FIG. 4B) and subsequent HF, which was defined as an ejection fraction (EF)<40% with severe anterior wall hypokinesis (see FIGS. 4C-4D) (Kumar et al. 2005, Michael et al. 1995). Third-order mesenteric resistance arteries within the splanchnic circulation were studied since these types of vessels are prime determinants of systemic vascular resistance (Christensen et al. 1993). Six weeks after surgery, myogenic tone was greater in the resistance arteries from HF mice compared to sham-control mice (see FIGS. 4E-4G). The increase in myogenic tone was not caused by vessel remodeling or changes in extracellular matrix constituents (Wan et al. 2013). Taken together, myogenic tone, measured in the absence of systemic factors such as short-acting circulating neurohormones and neural inputs, is markedly increased in HF.

### Example 4

### BK Currents are Reduced in HF.

Strong hyperpolarizing currents are required to prevent excessive contraction in response to intraluminal pressure and vasoconstrictors, and for relaxation in response to vasodilators. BK channels are especially important contributors to these hyperpolarizing currents by virtue of their large conductance and Ca²⁺ sensitivity (Nelson et al. 1995). In smooth muscle, BK α associates with its (31 regulatory subunit, priming the channel for activation by Ca²⁺ sparks from the nearby ryanodine receptors (see FIG. 3) (Brenner et al. 2000, Knaus et al. 1994, Cox et al. 2000, Bao et al. 2005). In the absence of the (31 subunit, BK channels do not sense the Ca²⁺ sparks, leading to increased myogenic tone and reactivity to norepinephrine (Brenner et al. 2000, Xu et al. 2011). In mice with HF, reduced expression and activity of BK α and β1 sensitize VSM cells to depolarization causing increased cytosolic Ca²⁺ concentration and myogenic tone (Wan et al. 2013). These changes correlate with human data: loss-of-function BK α polymorphisms are linked to increased systolic blood pressure and are weakly associated with increased risk of MI, whereas a gain-of-function β1 polymorphism (E65K) is associated with decreased incidence of diastolic hypertension, MI, and stroke (Tomas et al. 2008, Jordan 2008, Fernandez-Fernandez et al. 2004, Senti et al. 2005, Kokubo et al. 2005, Via et al. 2005). Reduced expression and/or function of BK channels contribute to elevated vascular tone in many, but not all animal models of aging, metabolic syndrome, diabetes and hypertension (Nystoriak et al. 2014, Phillips et al. 2005, Dong et al. 2009, Rusch 2009, Liu et al. 2009, Nieves-Cintron et al. 2008, Amberg et al. 2003a, Amberg et al. 2003b, Bagi et al. 2005, Lagaud et al. 2001, McGahon et al. 2007a, McGahon et al. 2007b, Lu et al. 2008, Dong et al. 2008, Yang et al 2012). In Ossabaw miniature swine with metabolic syndrome for example, although BK α and β1 subunit expression are increased, BK currents are reduced, likely due to altered cell surface localization (Rusch 2009, Borbouse et al. 2009). Thus, determining both BK channel expression and function are critically important. These findings offer the first evidence that in systolic HF, the electrical homeostasis of VSM cells is altered, substantially due to reduced expression and function of BK channels.

### Molecular Basis for Reduced BK Channel Expression and Function.

The increased circulating hormones and cytokines, and formation of ROS are important contributors to the HF syndrome, and are the likely effectors, at least in part, of the reduced expression and function of BK channels. There is precedent for this: for instance, the expression of BK subunits is transcriptionally regulated by circulating angiotensin II (Ang II), aldosterone, and the cytokine IL-1β (Amberg et al. 2003a, Amberg et al. 2003b, Layne et al. 2008, Nieves-Cintron et al. 2007, Ambroisine et al. 2007, Gao et al. 2010). BK β1 expression is a direct target of serum response factor-myocardin transactivation (Long et al. 2009). Forced expression of myocardin increased BK β1 expression, and knockdown of serum response factor decreased BK β1 expression. Although mice with HF had no change in the mRNA expression of serum response factor and myocardin compared to sham controls (assessed using an Affymetrix GeneChip Gene 1.0 ST Array, see below), HF may cause changes in the post translational mechanisms regulating myocardin's protein stability or transcriptional activity (Xie et al. 2009, Morita et al. 2014, Mack 2011, Choi et al. 2010). Miano and colleagues also showed that BK α mRNA expression was not dependent upon myocardin, implying distinct transcriptional control mechanisms of the two BK subunits (Long et al. 2009). The loss of protection from depolarizing influences in the vasculature of HF animals is analogous to altered expression and function of K⁺ channels in cardiomyocytes occurring in conditions such as HF and cardiac hypertrophy (Tomaselli et al. 2004).

Inhibitors of circulating hormones may restore normal expression and function of BK α and β1. If normalizing BK channel expression/function is therapeutic, the transcriptional regulation of BK subunits are explored. BK β1 association with BK α may also be dynamically regulated, based upon a recent study demonstrating that antegrade trafficking of β1 subunit-containing Rab11A-positive recycling endosomes and subsequent association with surface BK α is the primary mechanism by which NO activates BK channels and induces vasodilation (Leo et al. 2014).

### Example 5

Decreased Vascular BK Channel Activity Reduces Survival and Heart Function after MI.

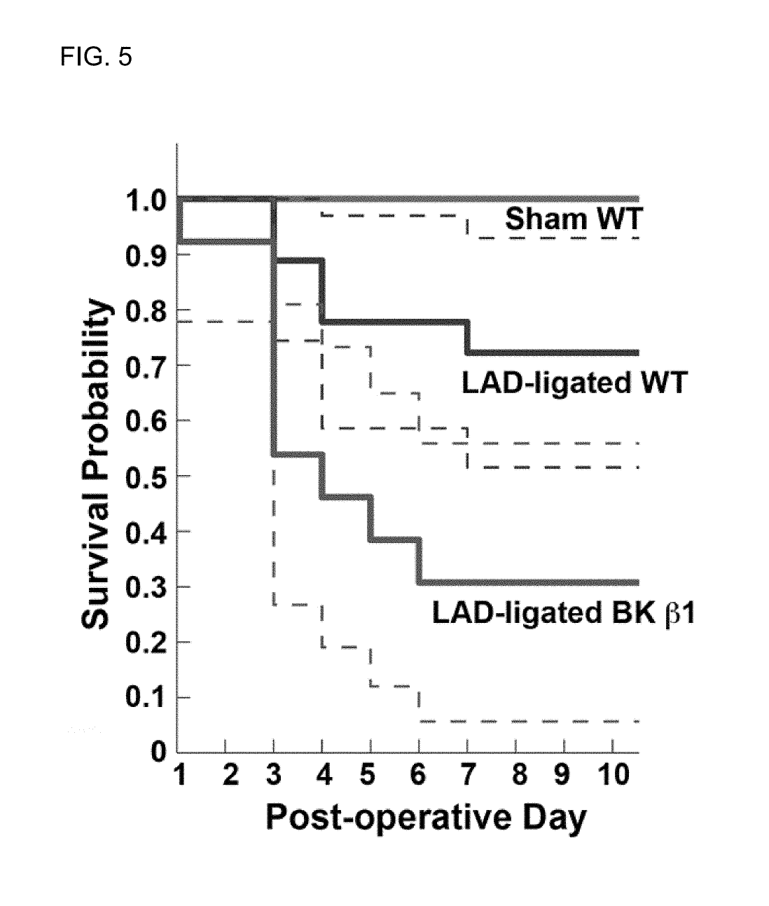

In the setting of acute myocardial ischemia, coronary vasodilation is an important means of enhancing oxygen delivery to threatened myocardium in order to limit infarct size. 10- to 12-week old male mice with deletion of BK β1 were found to have markedly reduced survival after LAD-ligation (see FIG. 5). The 10-day mortality of the BK β1 null mice was markedly higher than that of the LAD-ligated WT mice, 72% vs. 28%. This phenotype is likely due to reduced Ca²⁺ sensitivity of BK α in VSM cells, which disables the channel's role as a mediator of negative feedback upon Ca²⁺ entry and contraction (Knaus et al. 1994, Bao et al. 2005). The BK β1 null mice were not known to have a striking vascular phenotype, save for mild hypertension (Brenner et al. 2000, Pluger et al. 2000).

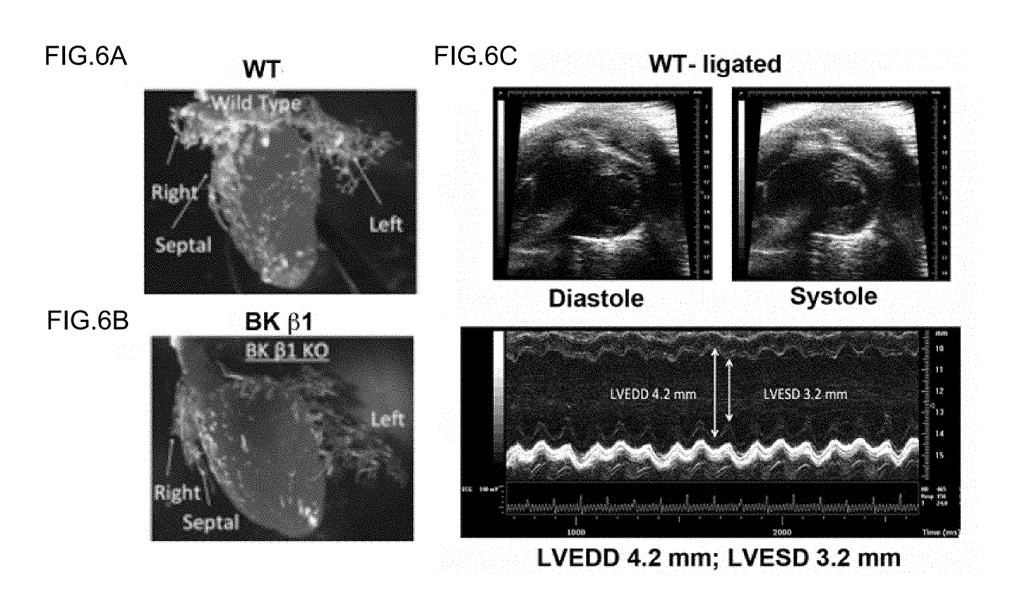

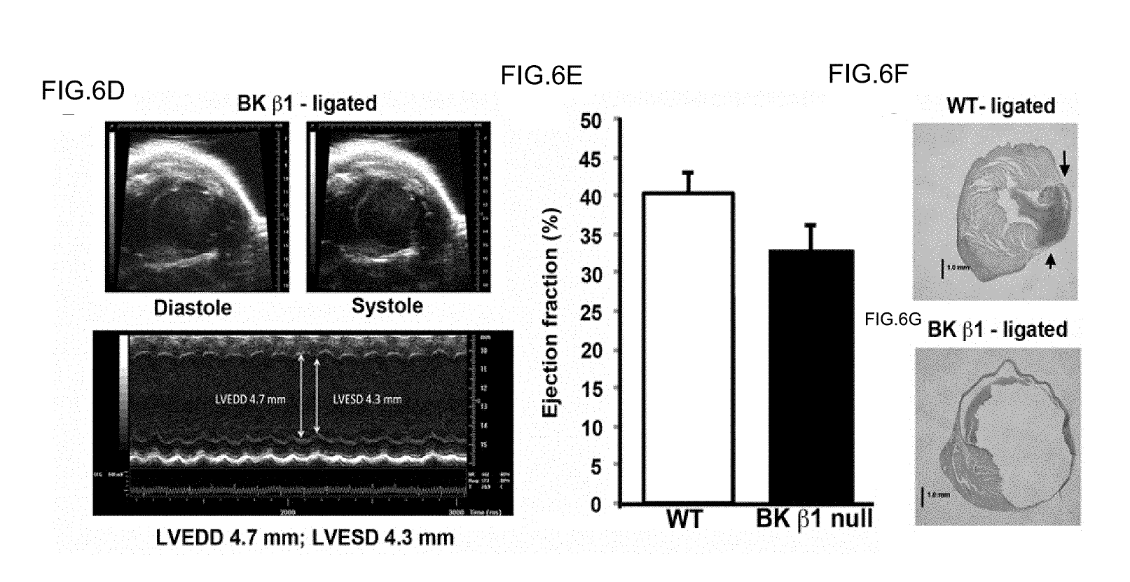

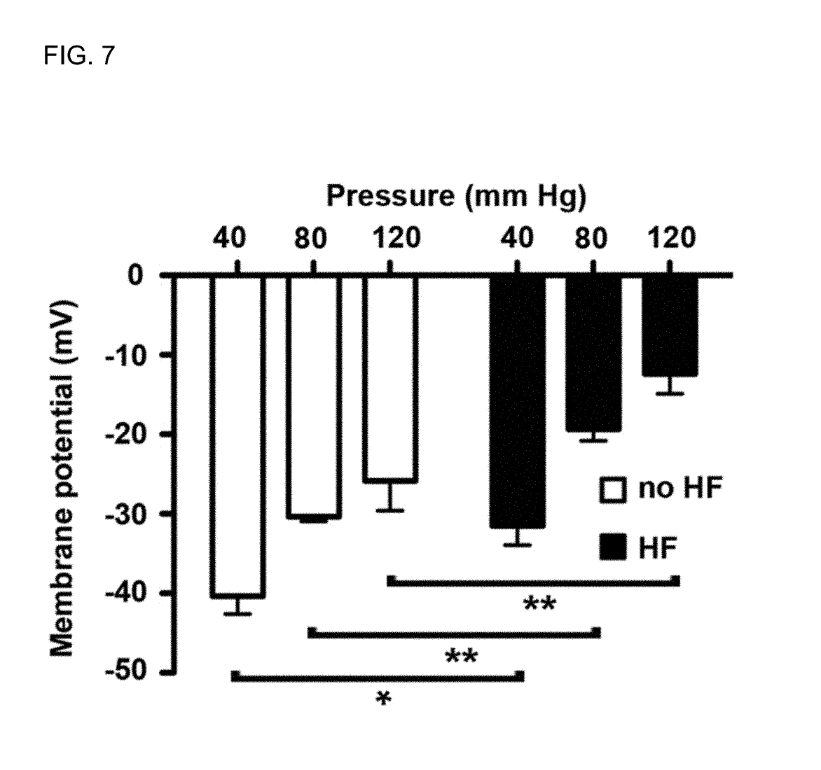

The coronary anatomy of WT and β1 null mice, assessed using plastic replica casts, were grossly indistinguishable, insofar as the number of major vessels were equal (see FIGS. 6A-6B). β1 null mice have normal cardiac function prior to LAD ligation, but showed reduced ejection fraction compared to WT mice 6 weeks after ligation (see FIGS. 6C-6E). This may be an underestimate because the mice that died soon after ligation were likely to have had poor cardiac function, but were not sonographically assessed. The hearts of β1 null mice exhibited larger infarctions and more severe aneurysmal dilatation than the hearts of WT mice (see FIGS. 6F-6G).

Since the sole validated function of BK β1 is to modulate the open probability of BK α in smooth muscle, and BK β1 is not expressed in adult cardiomyocytes, these findings reveal an important protective role for vascular BK channels in the peri-MI period (Brenner et al. 2000, Long et al. 2009, Jiang et al. 1999, Behrens et al. 2000). Activating BK channels may be a novel approach to treat coronary spasm and myocardial hypoperfusion after MI.

### Example 6

### Enhancing Endothelial-Induced Hyperpolarization of Smooth Muscle to Attenuate Increased Myogenic Tone.

Whereas endothelial-derived vasodilators, such as P450-derived epoxyeicosatrienoic acids (EET), NO, prostacyclin, lipoxygenase products and hydrogen peroxide, hyperpolarize and relax VSM cells by activating BK channels, BK channels are not required for EDHF-mediated vasodilation (Feletou 2009, Larsen et al. 2006, Liu et al. 2011). Increasing SK3 and IK1 currents by transgenic or pharmacological approaches decreased myogenic tone, increased acetylcholine-induced relaxation of rat cremaster arterioles, and restored the attenuated EDHF-type relaxation in mesenteric arteries from Zucker diabetic rats (Taylor et al. 2003, Brondum et al. 2010). Activating SK3 and IK1 channels induced dilation and increased coronary flow in Langendorff-perfused rat hearts (Mishra et al. 2012). Enhancing EDHF by activating endothelial SK3 and IK1 channels is an innovative and unique approach to circumvent the dysfunctional control of the VSM membrane potential caused by decreased BK channel expression and function in HF. SKA-31, a specific activator of SK3 and IK1 channels, caused dilation of mesenteric resistance vessels isolated from mice with HF. Indirectly hyperpolarizing the underlying VSM cells through enhancing EDHF (see, e.g., FIGS. 1A-1B and FIG. 2) rescued the increased mortality in BK β1 null mice (see FIG. 5) and improve LV function in WT (see FIGS. 4A-4G) and β1 null mice (see FIGS. 6A-6G) after MI.

### Example 7

VSM Plasma Membrane Potential is Depolarized in Mesenteric Arteries of Mice with HF.

The plasma membrane potential of VSM cells was directly measured in intact vessels by impaling them through the adventitia with glass microelectrodes. At three intraluminal pressures, the degree of depolarization was significantly greater in vessels from mice with HF than sham-controls (see FIG. 7). Since membrane depolarization leads to opening of voltage-gated Ca²⁺ channels, cytosolic Ca²⁺ was measured within the vessel wall of pressurized vessels using ratiometric measurements of fura-2 fluorescence. Pressurized vessels from HF mice exhibited a higher concentration of (Ca²⁺)ᵢ than those from sham mice, consistent with the depolarized membrane potential.

### Example 8

### Transcriptional Changes of Vascular Ion Channels in HF.

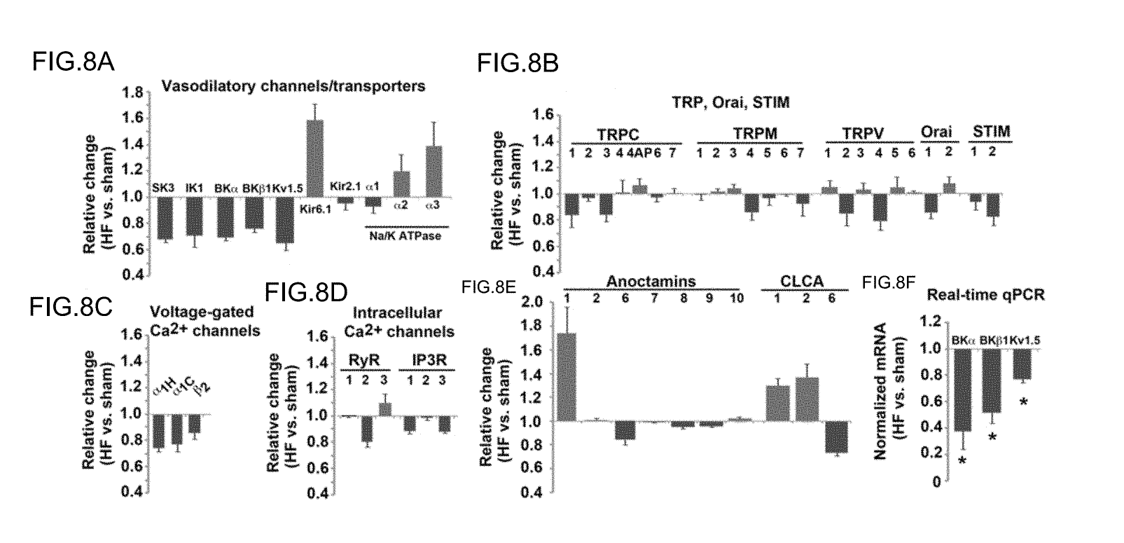

RNA, from third-order mesenteric arteries of mice 6 weeks after sham or LAD-ligation, was hybridized to an Affymetrix GeneChip Gene 1.0 ST Array. For the primary analysis, in which the hypotheses were pre-specified for candidate ion channels and pumps, the type I error rate was controlled at 0.01. The conclusions are: (1) of the 88 detectable K⁺ channels and related regulatory proteins, 4 K⁺ channels were reduced by >1.4-fold, SK3, IK1, BK α and K_{V}1.5 (see FIG. 8A, P<0.01). BK β1 was reduced by >1.3-fold. LRRC26 (BKβ1) was unchanged. The reductions were confirmed by real time qPCR: BK α and β1 were decreased to 36% and to 50.4% of sham respectively, and K_{V}1.5 was reduced to 75% of sham (see FIG. 8F). 16.1 (KCNJ8) mRNA was significantly increased by 1.6-fold, but this should reduce myogenic tone. (2) The depolarization of the smooth muscle could also be due to increased Na⁺, TRP and/or Cl⁻ channels (Large et al. 1996). Na⁺ and TRP mRNA levels were not were significantly altered. TRPV4, α_{1C} (L-type) and α_{1H} (T-type) were decreased by 1.3-fold. TRPV4 is required for shear force- and endothelial-dependent vasodilation (Earley et al. 2005, Earley et al. 2009, Sonkusare et al. 2012). The mRNA of TMEM16A/anoctamin 1, which forms Ca²⁺-activated Cl⁻ channels, was significantly increased by >1.7-fold in HF mice (see FIG. 8E, P<0.01). This change could account, in part, for the depolarization of VSM cells. (3) The mRNA levels of RyR and inositol 1,4,5 trisphosphate receptor (IP3R) were not significantly altered (see FIG. 8D).

### Example 9

Decreased Expression and Function of Vascular BK Channels in Mice with HF.

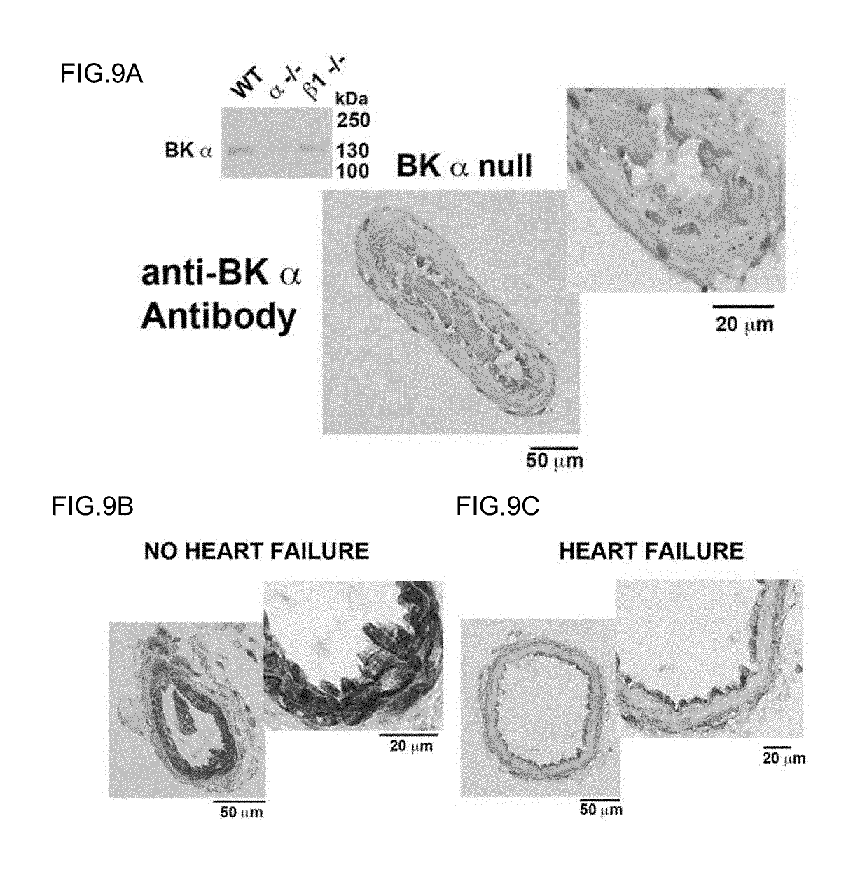

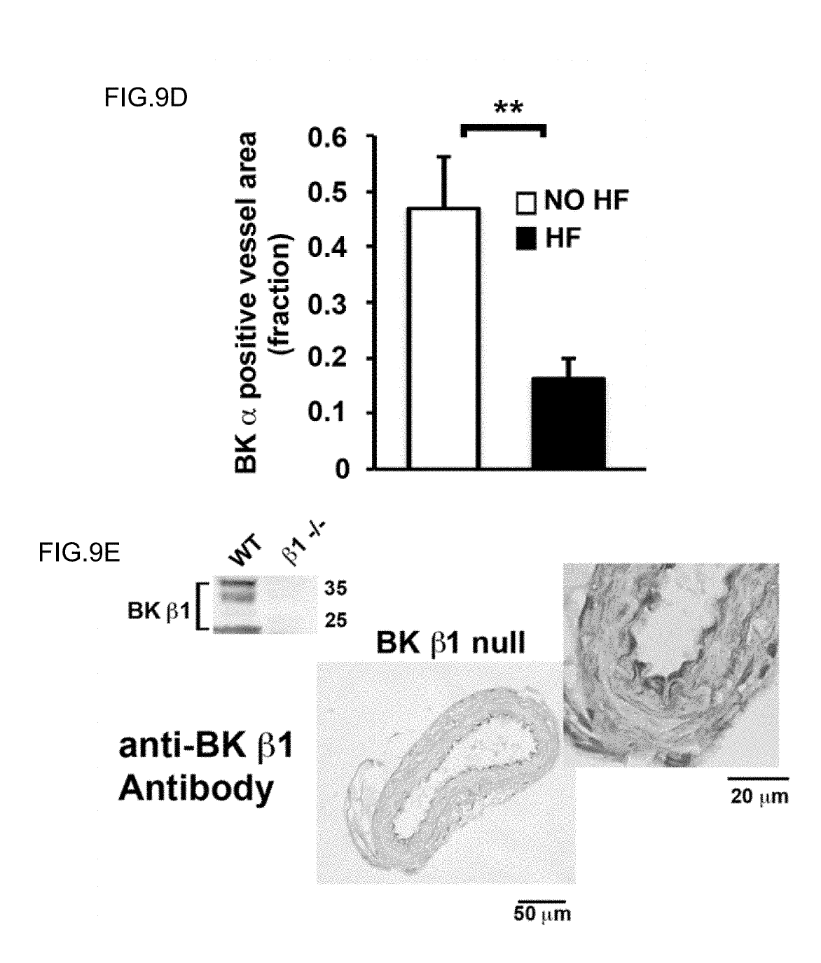

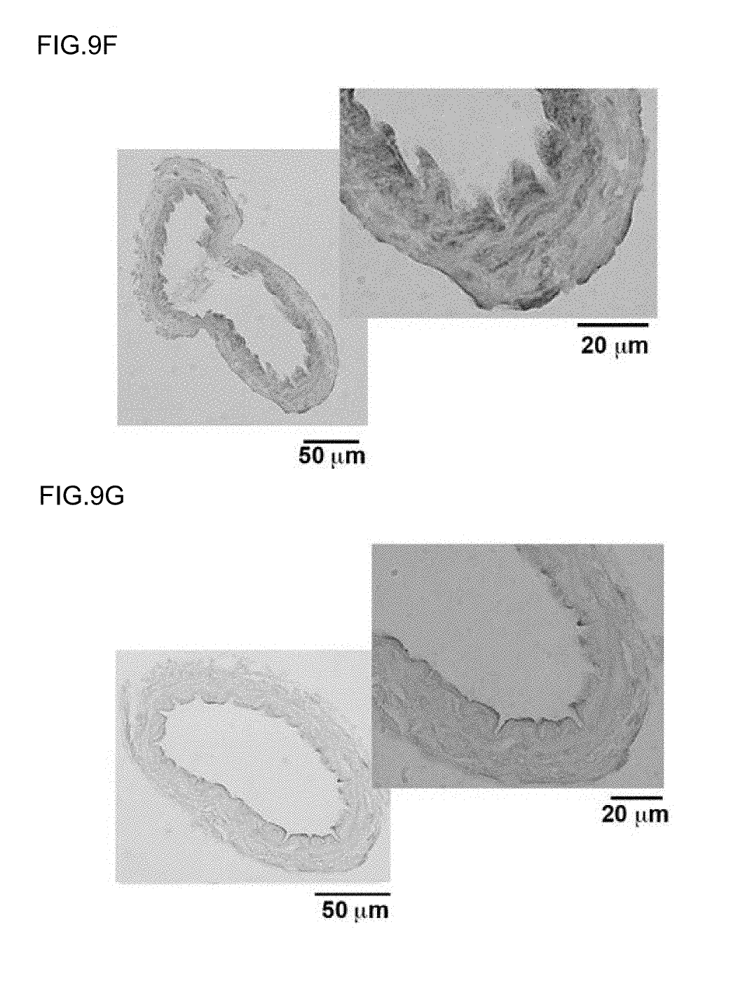

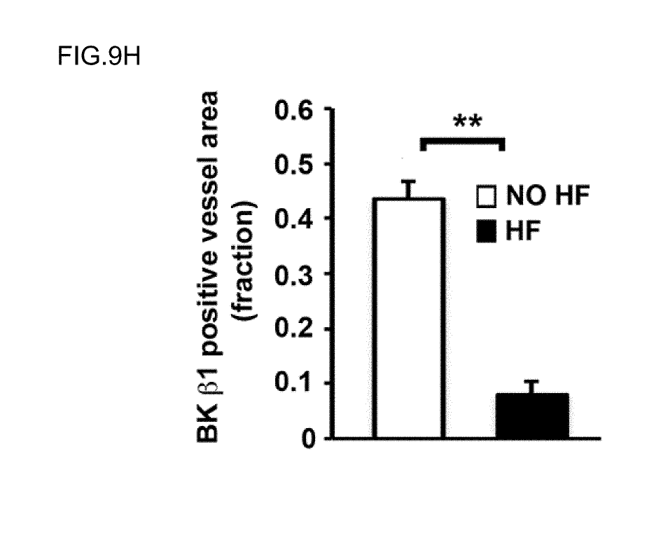

Immunohistochemistry (IHC) was used to semi-quantitatively assess the expression of BK α and β1 in the tunica media layer of mesenteric arteries. The antibodies were deemed specific based upon the failure to detect BK α and β1 in immunoblots and IHC of arterial extracts of BK α and β1 null mice, respectively (see FIGS. 9A & 9E). Many other groups have demonstrated the specificity of these antibodies to varying degrees (Amberg et al. 2003, Singh et al. 2013, Borbouse et al. 2009, Wulf et al. 2009, Pyott et al. 2007, Pluznick et al. 2005, Meredith et al. 2006, Grimm et al. 2007). BK α (see FIGS. 9B-9D) and β1 (see FIGS. 9F-9H) expression was markedly decreased in mesenteric vessels from mice with HF relative to controls (Wan et al. 2013). In HF vessels, the mean area of BK α and β1-DAB-immunostaining was reduced by 65% and 82% respectively, compared to controls (see FIGS. 9D and 9H).

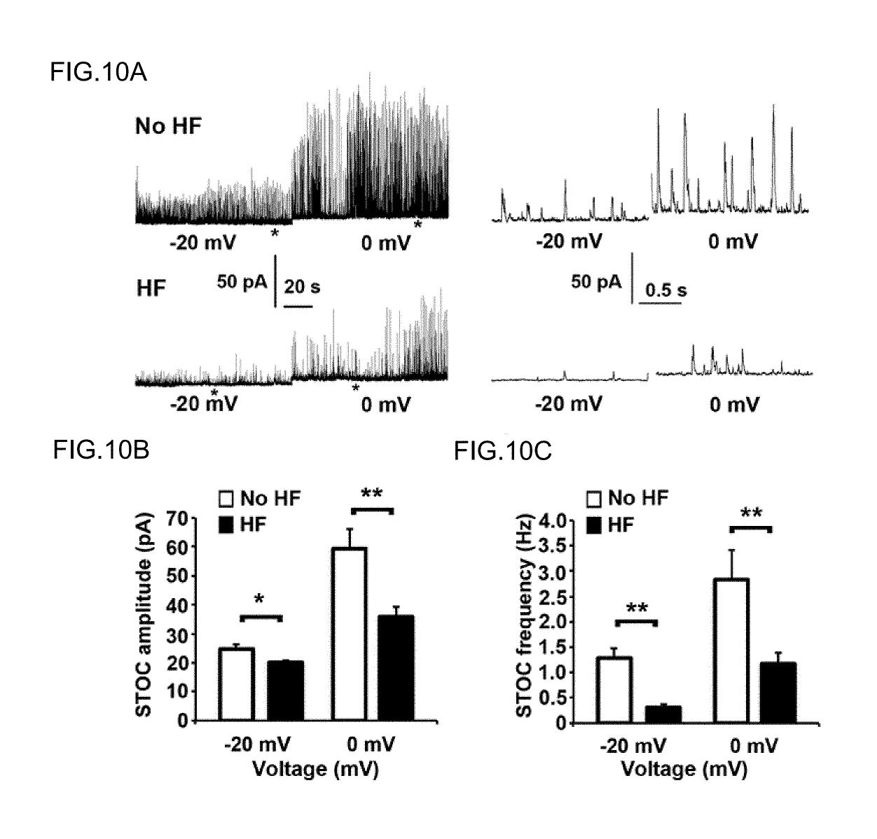

Spontaneous transient outward currents (STOCs), the simultaneous openings of BK channels in response to localized Ca²⁺ sparks, were measured in freshly isolated third-order mesenteric VSM cells (see FIG. 10A). Depolarization increased the frequency and amplitude of STOCs from both sham-control and HF mice (see FIG. 10A), but both amplitude (see FIG. 10B) and frequency (see FIG. 10C), at −20 mV and 0 mV, were significantly reduced in VSM cells from HF mice (Wan et al. 2013). These findings cannot be attributed to changes in the size of VSM cells, since the cell capacitance did not differ significantly (HF: 16.1+0.6 pF vs. control: 14.8+0.1 pF, p=NS) and normalizing each cell for its capacitance did not change the overall results. The diminished STOC-frequency is likely due to the marked decrease in β1 expression, which reduces the sensitivity of BK channels to activation by Ca²⁺. The reduced STOC-amplitude is likely due to the reduction in both BK α and β1 expression, although post-translational modifications of BK channels may also contribute. These data show that HF induces an electrical remodeling within the vasculature, leading to increased VSM depolarization and vessel contraction.

### Example 10

### Reduced BK Channel Expression and Function are Sufficient to Cause Increased Myogenic Tone.

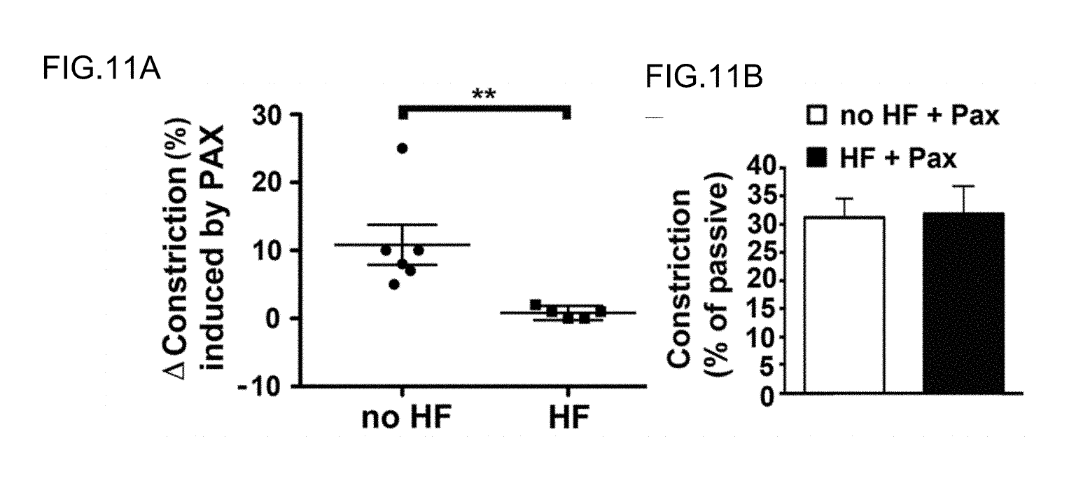

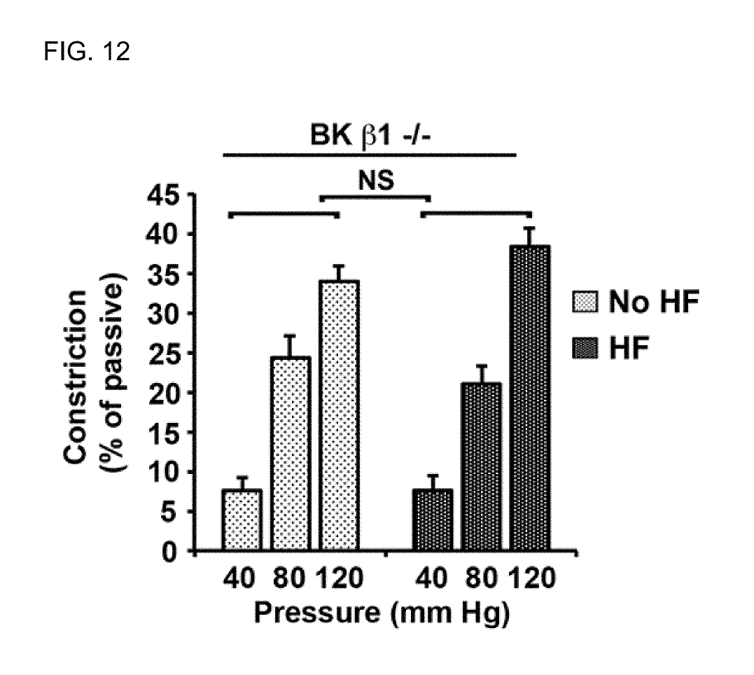

In mesenteric arteries isolated from mice without HF, paxilline, a specific BK channel blocker, increased constriction by 10.9%, a relative increase of 51.9% in myogenic tone (see FIG. 11A). If HF alters myogenic tone by reducing BK channel expression and currents, then the effect of paxilline should be reduced. Confirming this hypothesis, it was found that in mesenteric arteries isolated from HF mice, paxilline increased constriction by 0.9%, a relative increase of only 3% (see FIG. 11A). Inhibiting BK channels with paxilline in sham-treated (no HF) control mice caused an increase in myogenic tone, approximating the increased myogenic tone of vessels isolated from HF mice (see FIG. 11B).

If the increased myogenic tone of HF was due to diminished BK currents secondary to reduced expression of BK channel subunits, HF should have a minimal effect on the already elevated myogenic tone in BK β1 null mice. To test this premise, the myogenic tone of sham-operated and LAD artery-ligated BK β1 null mice was compared. Despite having severely reduced LV function (see FIGS. 6A-6G), HF did not further increase the already elevated myogenic tone in the BK β1 null mice (see FIG. 12), unlike WT mice (see FIG. 4G). Taken together, these data show that reduced BK currents within VSM cells are substantially responsible for the enhanced pressure-induced membrane depolarization and myogenic constriction observed in resistance vessels of mice with HF.

### Example 11

Characterization of the Vasoreactivity and Electrical Profiles of Coronary Septal Arteries and Third-Order Mesenteric Resistance Arteries Early after MI and During the Progression to Severe HF.

The myogenic tone of mesenteric vessels were measured, 4-months post-MI. The myogenic tone in mesenteric vessels was 36%, 42% and 43% at 40, 80 and 120 mm Hg, respectively, which was greater than at 6-weeks post-MI for 40 and 80 mm Hg. These results are consistent with the hypothesis that as HF worsens, vascular resistance increases.

Four time-points are used: 2 days, 2 weeks, 6 weeks and 4 months post-MI or post-sham operations. LV function is assessed by echocardiography, and post-mortem cardiac histopathology and lung to body weight ratio. Two vascular beds are studied: (i) coronary septal arteries and (ii) third-order mesenteric arteries. Coronary septal arteries, which are identified as described (Sankaranarayanan et al. 2009, Edwards et al. 1998, Davis et al. 2012), are not perturbed during LAD-ligation because they are branches of the right coronary artery (see FIGS. 5A and 1 in Kumar et al. 2005) and not the LAD as they are in humans. Myogenic tone and VSM Ca²⁺ concentration are determined simultaneously using the Ionoptix system (Wan et al. 2013). The VSM cell membrane potential is measured in intact vessels as shown in FIG. 7.

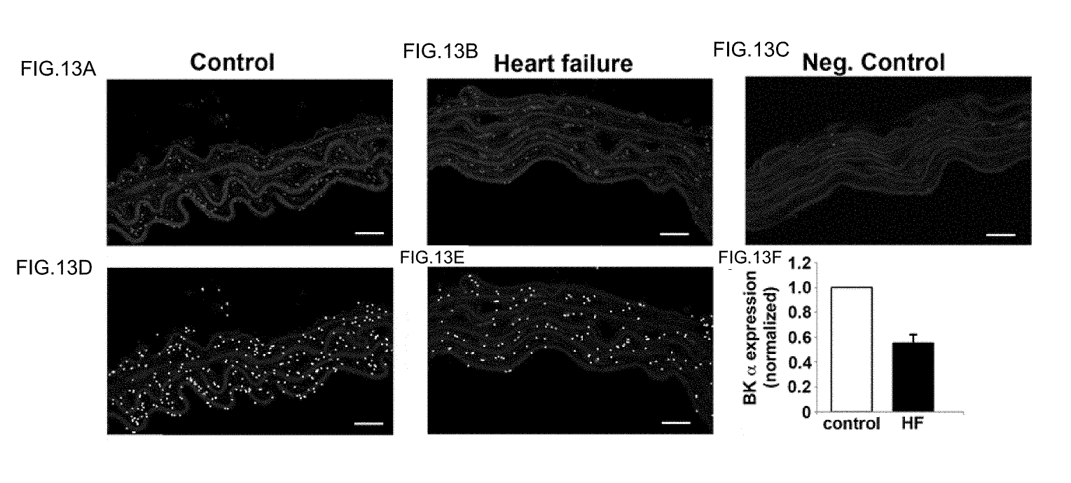

Real-time qPCR, semi-quantitative immunoblotting and immunohistochemistry are performed (using DAB-IHC and Duolink—see FIGS. 9A-9H and FIGS. 13A-13F) to assess the expression of Ca_{V}1.2, Kir2.1, K_{V}1.5, TMEM16A, TRPV4, which may act as the EET receptor, and TRPC6, TRPM4, and TRPP1, which have been implicated in the myogenic response (Earley et al. 2010). Immunoblots are normalized to actin.

Duolink has >100-times higher signal:noise ratio than standard immunofluorescence (0-link manual, page 8). Frozen sections of mice aortas were incubated first with anti-BK α monoclonal antibody, and then with two distinct anti-mouse secondary antibodies. These secondary antibodies are known as proximity ligation assay (PLA) probes, and each is attached to a unique short DNA strand. When the two PLA probes are in close proximity (<40 nm) to one another, the DNA strands interact and are amplified several-hundredfold by a polymerase. Each distinct spot represents either a single BK α tetramer or closely apposed tetramers. Using the Duolink image tool, individual spots were automatically identified (see FIGS. 13D-13E) and counted. The bar graph (see FIG. 13F) indicates the reduction in the number of “spots” per field in the aorta of HF mice compared to that of the control, consistent with the reductions found in the mesenteric vessels using standard IHC (see FIGS. 9A-9H).

Mesenteric and coronary VSM cells are isolated from HF and sham mice for in-depth electrophysiological analysis: (a) voltage-dependent Ca²⁺ currents are measured using whole-cell technique (Navedo et al. 2007). K⁺ currents are inhibited using 1 μM paxilline and 10 mM 4-aminopyridine, and CsCl in the pipette. (b) K_{V} channels are measured using whole-cell voltage clamp. BK channels are blocked with paxilline. (c) Ca²⁺-activated Cl⁻ currents (TMEM16a) are measured using whole-cell configuration with Cs⁺ replacing K⁺ in the intracellular solution (Manoury et al. 2010, Hartzell et al 2009). Cells are dialyzed with solutions containing either 17 or 600 nM free Ca²⁺. (d) Mechanosensitive cation channels (TRP) are recorded using cell-attached configuration, applying mild negative pressure of 7.5-15 mm Hg. The bath and pipette solution contain 140 mM CsCl (Ozgen et al. 2007).

If the Ca²⁺-activated Cl⁻ currents mediated by TMEM16a are increased in HF, for example, reducing these currents using an inhibitor, T16Ainh-A01 (aminophenylthiazole), may normalize myogenic tone in vitro (Bulley et al. 2012, Davis et al. 2012). A similar approach is used for mechanosensitive (TRP) channels.

### Example 12

### Identification of the Molecular Mechanisms Underlying HF-Induced Reductions in Both the Expression and Function of BK Channels.

The reduction in STOCs (see FIGS. 10A-10C) may be due to changes in the number of BK channels and/or open probability. Changes in open probability may be due to reduced BK β1 expression, post-translational modifications and/or mislocalization of BK channels (no longer close to sources of Ca²⁺, such as ryanodine receptor or Ca²⁺ channels, which can be assessed, to some extent, semi-quantitatively using IHC/Duolink and co-immunoprecipitation (Liu et al. 2004). These experiments are focused on VSM cells isolated from third-order mesenteric vessels 6 weeks post-MI and sham-controls.

### Example 13

### Role of Post-Translational Modifications of BK Channels by Phosphorylation and Reactive Oxygen Species (ROS) in Reducing BK Currents.

BK channels integrate the signals of multiple kinases activated by classical neurohormonal pathways, including the sympathetic nervous system and RAAS. PKA and PKG phosphorylation increase BK channel activity, whereas PKC and c-Src phosphorylation decrease BK channel activity (Schubert et al. 2001, Alioua et al. 2002). PKC and c-Src inhibitors are used, anticipating that if the channels are inhibited because of PKC or c-Src phosphorylation in VSM cells from HF mice, BK currents increase upon inhibition of PKC or c-Src. Similarly, oxidative stress within the vessel wall may be responsible for depolarization and reduced amplitude and frequency of STOCs in VSM cells from HF mice. Because of the non-specificity of Nox inhibitors, two inhibitors are tested: diphenylene iodonium (Lu et al. 2010) and apocynin, anticipating that if BK channels are inhibited due to oxidation in VSM cells from HF mice, BK currents increase after NAPDH oxidase inhibition. The results are compared to VSM cells isolated from sham mice, as PKC, c-Src or Nox antagonists should have minimal effects on these channels.

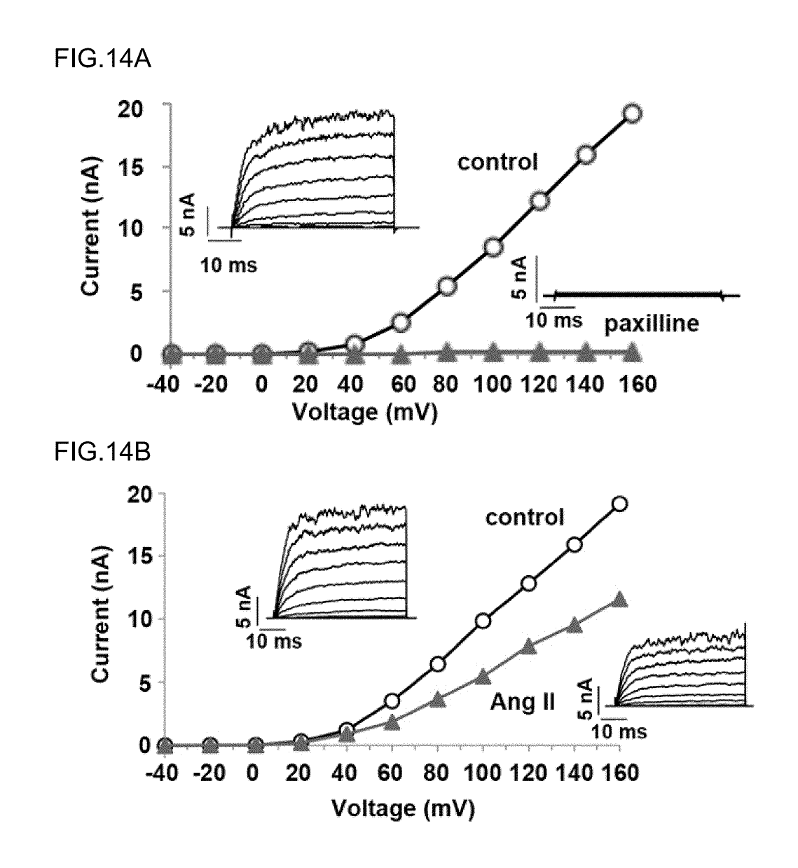

Conversely, Ang II (2 μM) or phenylephrine (10 μM) may differentially modulate channel function in VSM cells isolated from HF mice. Ang II inhibits BK channel function through its effects on PKC, c-Src and Nox (Lu et al. 2010). Ang II causes less inhibition if BK channels are already modified by PKC, c-Src or Nox pathways, as compared to the effect on BK channels from control VSM cells, in which Ang II suppressed BK current by 50% (see FIG. 14B).

Two determinants of BK activity are used: STOCs and whole-cell current density. BK currents are a very large fraction of the total K⁺ currents in freshly isolated mesenteric VSM cells (see FIG. 14A). To measure the effects of PKC, c-Src and Nox inhibitors on STOCs, the VSM cells are incubated for 1-hour with one of the following: Bisindolylmaleimide I, HCl, the PKCα, PKCβ, PKCδ, PKCε and PKCγ inhibitor, Lavendustin A (10 μM), a c-Src inhibitor (or as a control, its inactive congener, Lavendustin B), diphenylene iodonium or apocynin, Nox inhibitors (Lu et al. 2010). PKCα and PKCδ may be the relevant PKC isoforms, which are further delineated using specific inhibitors.

### The Reduction in Expression and Function of BK Channels Due to Activation of Renin-Angiotensin-Aldosterone System (RAAS).

Ang II, aldosterone and cytokine IL-1β, which are elevated in HF, are known to decrease the expression of BK α and β1 mRNA (Amberg et al. 2003a, Amberg et al. 2003b, Layne et al. 2008, Nieves-Cintron et al. 2007, Ambroisine et al. 2007, Gao et al. 2010). Spironolactone prevents the aldosterone-induced decrease in BK α mRNA in cultured rat VSM cells (Ambroisine et al. 2007). The activated RAAS and neurohumoral system is hypothesized to be responsible for the reduction in BK α and β1 mRNA expression. It will be determined whether in vivo administration of spironolactone and lisinopril can restore BK α and β1 expression and normalize myogenic tone. Angiotensin converting enzyme and aldosterone antagonists are mainstays of HF therapy. Changes in myogenic tone by these drugs may be due to their effects on transcriptional regulation of BK α and β1, and/or effects on channel gating.

Three groups of mice, each with 10 animals are used for this study. Two groups of mice undergo LAD ligation and the third group undergo sham ligation. After 6 weeks, LV function is assessed by echocardiography. For the HF groups, only those mice with EF less than 40% are used. One LAD-ligated/HF group is treated with both lisinopril (66 mg/L-Sigma) and spironolactone (250 mg/L-Sigma) in the drinking water. This combination has been used in mice and the concentrations are in the effective range for mice: 10 mg/kg/day for lisinopril and 37.5 mg/kg/day for spironolactone (Thomas-Gatewood et al. 2011). Five mice in each group are euthanized at 1 week and 3 weeks post-initiation of the drugs. LV function is assessed by echocardiography prior to euthanasia. Endpoints will include BK α and β1 expression in mesenteric and coronary vessels, assessed using real time qPCR, immunohistochemistry and immunoblot, and the myogenic tone of mesenteric vessels. Electrophysiological studies of isolated VSM cells from mesenteric and coronary vessels are performed if myogenic tone is normalized.

PKC phosphorylation of BK channels not only reduces BK currents, but also renders the channels insensitive to PKA and PKG stimulation (Zhou et al. 2010). If HF renders the BK channels relatively resistant to vasodilators that are dependent upon PKA and PKG signaling, the vasodilators sodium nitroprusside, salbutamol or milrinone are applied to VSM cells. If the channels are relatively resistant to PKA or PKG-stimulation, the PKC, c-Src or Nox inhibitors may normalize the response of BK channels to PKA- and PKG-dependent vasodilators. These studies will explore the recent finding (Leo et al. 2014) that BK β1 association with BK α may be dynamically regulated, and that NO activates BK channels by inducing the association of BK α and β1 at the surface membrane.

There are important differences between the hearts of mice and larger animals, especially in regards to metabolic and heart rate. HF frequently develops insidiously in humans over several years, whereas in most animal models, it occurs subacutely over weeks to months. Human vessels are obtained from HF patients (e.g. heart transplant recipients or HF patients undergoing surgical procedures such as ICD implantation) to determine whether HF causes elevation of myogenic tone and reduced expression of BK subunits.

### Example 14

### Generation of Tg Mice.

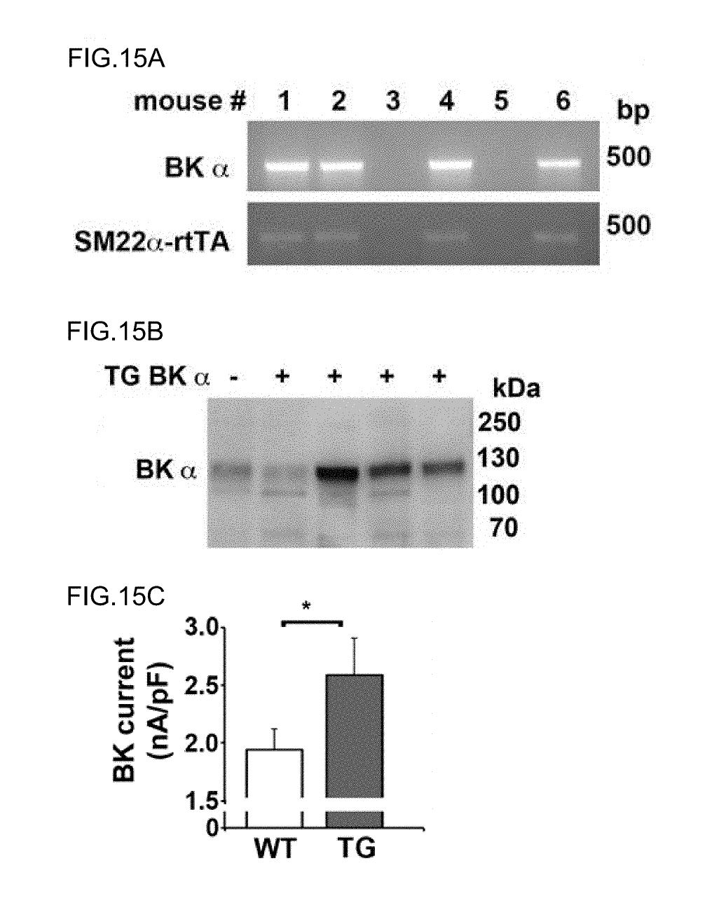

Doxycycline-inducible, VSM cell-specific BK α-expressing Tg mice were created. Founders were identified and were crossed with SM22α-rtTA mice to create double Tg mice (see FIG. 15A). The SM22a promoter fragment drives transcription limited to arterial smooth muscle (Bernal-Mizrachi et al. 2005, Moessler et al. 1996, West et al. 2004). Four founder lines were tested for protein expression after doxycycline, and three demonstrated increased BK α expression (see FIG. 15B). A 33% increased density of BK currents was found in mesenteric VSM cells in the Tg mice.

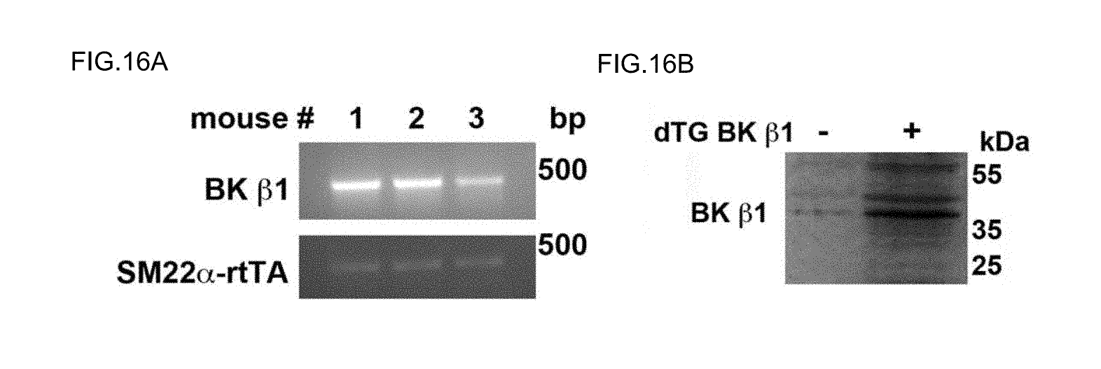

Doxycycline-inducible VSM-specific BK β1-expressing Tg mice were also created. Multiple founders were identified and these mice were also crossed with SM22α-rtTA mice, yielding double Tg mice (see FIG. 16A). Feeding the mice doxycycline increased BK β1 expression in VSM cells (see FIG. 16B). No adverse effects of expressing BK α or β1 subunits were detected.

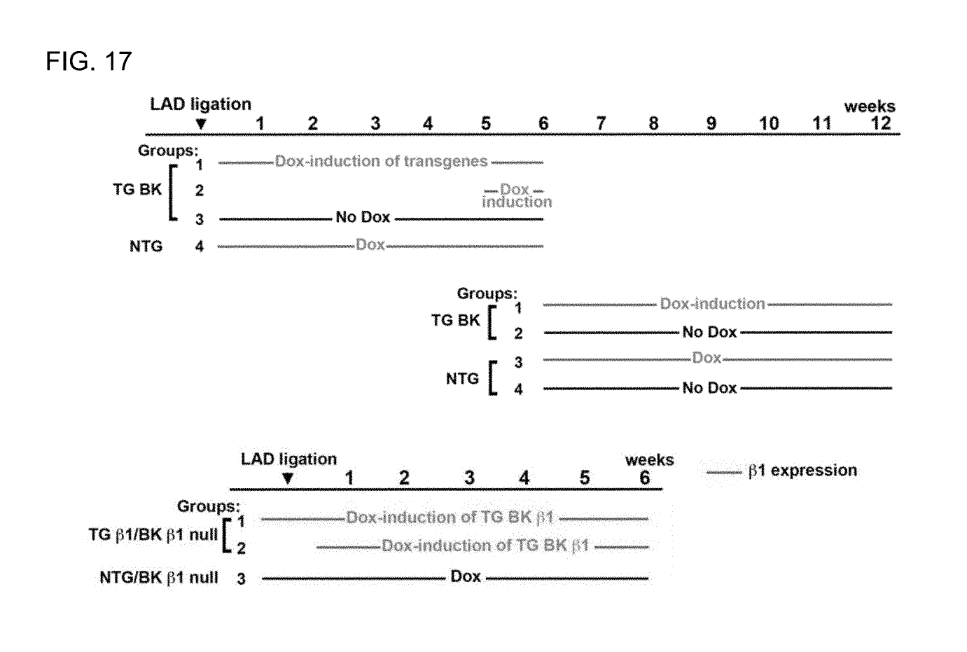

For both BK α and BK β1, there are four groups: Group 1: BK α or β1 expression starting 72 hours after LAD-ligation and continued to 6 weeks; Group 2: BK α or β1 expression starting 5 weeks after LAD-ligation; Group 3 (control): No doxycycline; Group 4 (control): Doxycycline in non-Tg (littermate). (see FIG. 17, top panel) All groups have 20 mice. Blood pressure is measured twice weekly using a tail cuff (Kent Scientific).

BK Current Density Measured in VSM Cells Isolated from Mice with HF.

The transgenes are likely not susceptible to HF-induced changes in transcription, unlike endogenous channels, confirmed by real time qPCR and immunohistochemistry. The primary endpoints are the extent of myogenic tone and the membrane potential of VSM cells in mesenteric arterial vessels. If the reduction in BK currents is responsible for the increased myogenic tone, restoration of BK current density (Groups 1 or 2) reduces myogenic tone and restores the normal membrane potential compared to Groups 3 and 4. These studies are complemented with determinations of Ca²⁺ concentration and STOCs. Secondary endpoints include LV function, assessed by echocardiography 2 days, and 1 and 3 weeks post-MI and by echocardiography and pressure-volume loops at 6 weeks post-MI. During post-mortem analysis, the severity of HF is inferred from lung weight, heart weight, and body weight ratios (Wan et al. 2013, Jones et al. 2003, Fraccarollo et al. 2008), and cardiac histopathological studies. Echocardiograms are performed using a Vevo 2100. High-frequency speckle tracking echocardiography derived LV ejection fraction, mass, volume, global and regional strain analyses are performed as described in Bhan et al. 2014. Infarct size is determined by 2,3,5-triphenyltetrazolium (TTC) staining. In addition, plasma BNP levels are determined by ELISA (Kamiya Biomedical). The study is sufficiently powered to detect an improvement in ejection fraction of 10% with a power of 0.8, a of 0.05 and standard deviation of 12. Mortality is tracked, but additional mice are required to detect a survival benefit: for instance, 58 mice in Group 1 are needed to achieve a power of 0.7 to detect a significant (a 0.05) improvement to 0.90 from 0.72.

Expression of K⁺ channels may worsen mortality or outcomes, perhaps due to hypotension. Adverse effects on blood pressure or survival have not been detected with a 30-50% increased expression of either BK α or β1 in mice prior to MI or HF. Titration and appropriate timing of expression of BK channels, however, may be important for the mice after MI and in HF.

Sufficiency of Increased Expression of BK Channels to Normalize Myogenic Tone and Improve LV Function in Mice with Pre-Existing HF.

For these experiments, it will be determined whether increasing BK currents when the HF syndrome is already established can reduce myogenic tone and improve cardiac function. This timing more closely mimics clinical practice. For both BK a or β1 Tg mice, there are four groups of mice, each with 10 animals (see FIG. 17, middle panel). Two groups are Tg mice and two groups are non-Tg littermates. All groups of mice undergo LAD-ligation. After 6 weeks, LV function is assessed by echocardiography. Only those mice with an ejection fraction less than 40% are used. One group of Tg mice and one group of non-Tg mice are then treated with doxycycline. The other groups do not receive doxycycline (control). LV function, the primary endpoint, is assessed by echocardiography weekly for 6 weeks, and at sacrifice, pressure-volume loops. Additional endpoints include myogenic tone and the severity of HF as described above. Blood pressure is monitored via tail cuff measurements.

### Sufficiency of Re-Expression of BK β1 in VSM to Rescue BK β1 Null Mice.

SM22α-rtTA/tetO-BK β1 mice (see FIGS. 16A-16B) are crossed with BK β1 null mice to create SM22α-rtTA BK β1/endogenous BK β1 null offspring. Doxycycline-induction of Tg β1 in mesenteric and coronary arteries is determined at 2, 5 and 10 days by IHC and immunoblot. Re-expression of β1 is confirmed by determining the conductance-voltage relationship in VSM cells and by assessment of myogenic tone in mesenteric and coronary vessels. In the background of endogenous BK β1 null, channels co-assembled with Tg BK β1 have a clear electrophysiological signature and are sensitive to the β1-specific agonist, DHS-1.

Three groups of mice are studied, each with 25 mice (see FIG. 17, bottom panel). The SM22α-rtTA β1/endogenous β1 null mice will be used in 2 groups: Group 1: doxycycline-induction of β1 before LAD-ligation. Group 2: doxycycline-induction of β1 starting 72 hours after LAD-ligation. Group 3: BK β1 null, non-Tg littermate mice treated with doxycycline before LAD-ligation, thus minimizing the effects of the mixed background of FVB/N×B6CBAF1×C57BL/6.

The operator and all data collection will be blinded. The primary endpoint is mortality: Group 1 and Group 2 vs. Group 3. Additional endpoints are: (a) Clinical severity of HF- to be assessed as described above; (b) Histopathological analysis of infarct size and LV dimensions; (c) Cardiac function—assessed using echocardiography 2 days and 1, 3, and 6 weeks after MI and by pressure-volume loops 6 weeks after MI. An improvement in survival can be detected to 60% in Group 1 or Group 2 vs. Group 3 with a power of 0.7 and a type I error probability of 0.05. An improvement in ejection fraction of 9% can be detected at day 2 with a power of 0.8, assuming 20 mice survive per group.

It is also important to determine whether expression of BK β1 in VSM cells prior to LAD ligation alters the ratio of infarct size (IS) to area-at-risk (AAR). Five additional mice in groups 1, 2 and 3 are used to assess the early effects of BK β1 expression. This ratio is expected to be reduced with BK β1 expression. AAR and IS are performed as previously described (Redfors et al. 2012, Moon et al. 2003, Takagawa et al. 2007). Seventy-two hours after LAD ligation, 2 ml of 5% Evans blue is injected into the RV chamber. The mice are subsequently euthanized with 4 ml of 0.5 M KCl. Serial 6-μM-thick cryostat sections are prepared. Well-perfused myocardium are outlined in blue. Unstained areas contain a combination of infarcted and unperfused, but viable myocardium (AAR). Sections are then incubated in TTC; AAR is stained red and dead tissue is white.

In addition to crossing the BK α and β1 Tg mice to co-express both BK α and β1, a doxycycline-inducible BK α-β1 Tg mouse line was developed using an internal ribosomal entry site. Co-expression of both BK α and β1 may be necessary to normalize myogenic tone and improve LV function. Expression of BK α and β1 subunits should increase the amplitude and frequency of STOCs. If the STOCs are not increased and membrane potential is not normalized, Ca²⁺ sparks and the ratio between Ca²⁺ sparks and STOCs are measured, which represents the coupling gain. HF may affect the coupling gain.

Rottlerin (mallotoxin) was identified as a potent activator of BK channels (Zakharov et al. 2005). Rottlerin increased BK currents in rodent and human VSM cells and decreased myogenic tone. These effects are β1 subunit-independent (Zakharov et al. 2005), an ideal property to circumvent the HF-induced reduction in BK β1 expression. Rottlerin markedly attenuated methacholine-induced airway hyperreactivity in two murine models of asthma without any apparent adverse effects (Goldklang et al. 2013). If experiments using Tg BK mice show a beneficial effect, the effects of rottlerin on myogenic tone and LV function will be studied. Plasma rottlerin levels were measured two hours after IP injection of 100 μg (5 μg/g) using LC/MS and a plasma level of 2.9 μg/mL was found (see Goldklang et al. 2013), which is more than the EC₅₀ for BK channel activation (Zakharov et al. 2005). Rottlerin is injected via IP injection 1 hour prior to LAD-ligation and its administration continued via daily IP injections for 1 week. If survival is improved, the experiments are continued for up to 6 weeks (without rottlerin injections) to determine if LV function is improved. If survival is improved but LV function is not improved, then experiments will be performed in which rottlerin is continued for the entire 6-week experimental period. Any beneficial effect of rottlerin may be multifactorial, since at relatively high concentrations, rottlerin can inhibit several cellular kinases including PKC (Gschwendt et al. 1994, Soltoff 2001, Soltoff 2007). Another BK agonist, NS11021 (Neurosearch) (Bentzen et al. 2007), is used for confirmation. If myogenic tone is normalized, and survival and LV function are not improved by VSM-specific re-expression of BK β1, but they are improved with pharmacological activation of BK channels, this suggests that non-vascular BK channels, perhaps in the cardiomyocyte mitochondria, might be responsible.

### Example 15

Indirect Hyperpolarization of VSM can Improve Survival and HF after MI in the WT and BK β1 Null Mice.

Another approach to correct the HF-induced depolarization of VSM cells is to enhance EDHF. Activation of endothelial SK3 and IK1 channels and the resulting hyperpolarization of endothelial cells is a crucial step for initiation of EDHF-mediated hyperpolarization of the VSM membrane potential and vasodilatation (Taylor et al. 2003, Edwards et al. 1998). SKA-31 (naphtha[1,2-d]thiazol-2-ylamine) is a potent activator of IK1 (EC₅₀ of 250 nM) and SK channels (EC₅₀ in low μM range) (Sankaranarayanan et al. 2009). SKA-31, which exhibits excellent selectivity for SK and IK1 channels, is not cytotoxic at concentrations up to 100 μM and has no acute toxicity in rats and mice. After administering SKA-31 (10 mg/kg) IP to mice, plasma concentrations were 5.5 μM at 2 h, 1.4 μM at 4 h and 500 nM at 24 h, of which 39% was plasma protein-bound and 61% was free, with higher concentrations in plasma than tissue. A single IP injection of 10 mg/kg SKA-31 lowered blood pressure by 4 mm Hg at 24 h, likely because the free plasma level of SKA-31 was 300 nM. A higher dose of 30 mg/kg lowered mean arterial pressure by 6 mm Hg in normal mice and by 12 mm Hg in mice with hypertension. Based upon these data, the initial dose of SKA-31 used was 30 mg/kg IP every 24 h.

### Activation of SK3 and IK1 Channels Cause Hyperpolarization of Endothelial Cell Membrane Potential.

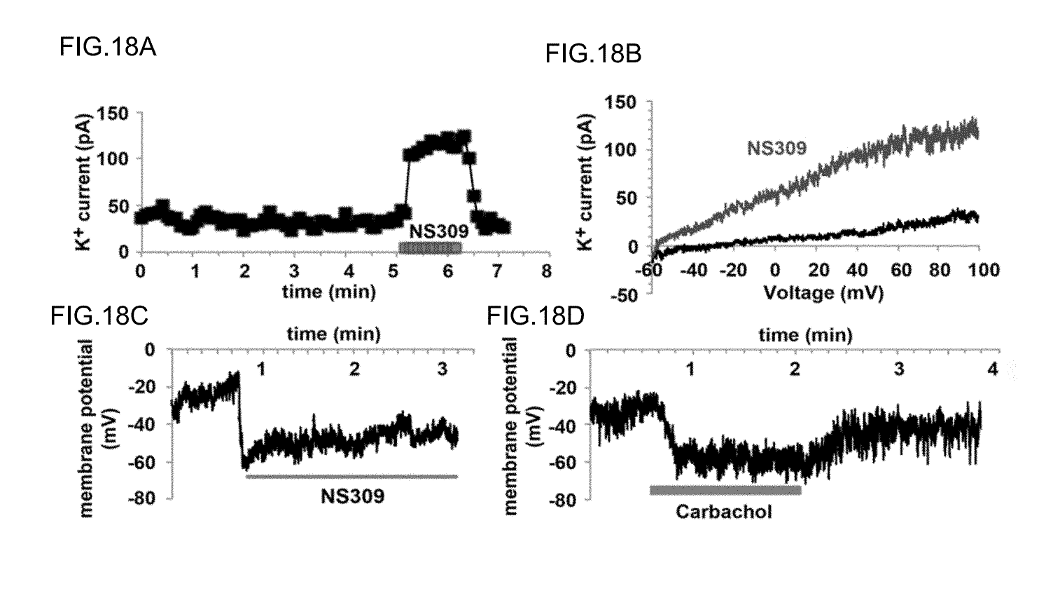

Endothelial cells were isolated from third-order mesenteric arteries of mice using enzymatic digestion followed by labeling the endothelial cells with CD34 antibody-labeled magnetic beads, collecting them using a magnet and then releasing them from the beads by competition. To measure both SK3 and IK1 currents, the cells were dialyzed with KCl-solution containing 3 μM free Ca²⁺ and currents elicited using voltage ramps and by the activator NS-309 (see FIGS. 18A-18B). Activation of SK/IK1 channels by NS309 caused hyperpolarization of the endothelial cell membrane potential, comparable to the effects of carbachol (see FIGS. 18C-18D). NS309, however, has an extremely short half-life, precluding its in vivo use.

Activation of SK3 and IK1 Channels Reduces Myogenic Tone in Mesenteric Resistance Vessels of Mice with HF and Controls.

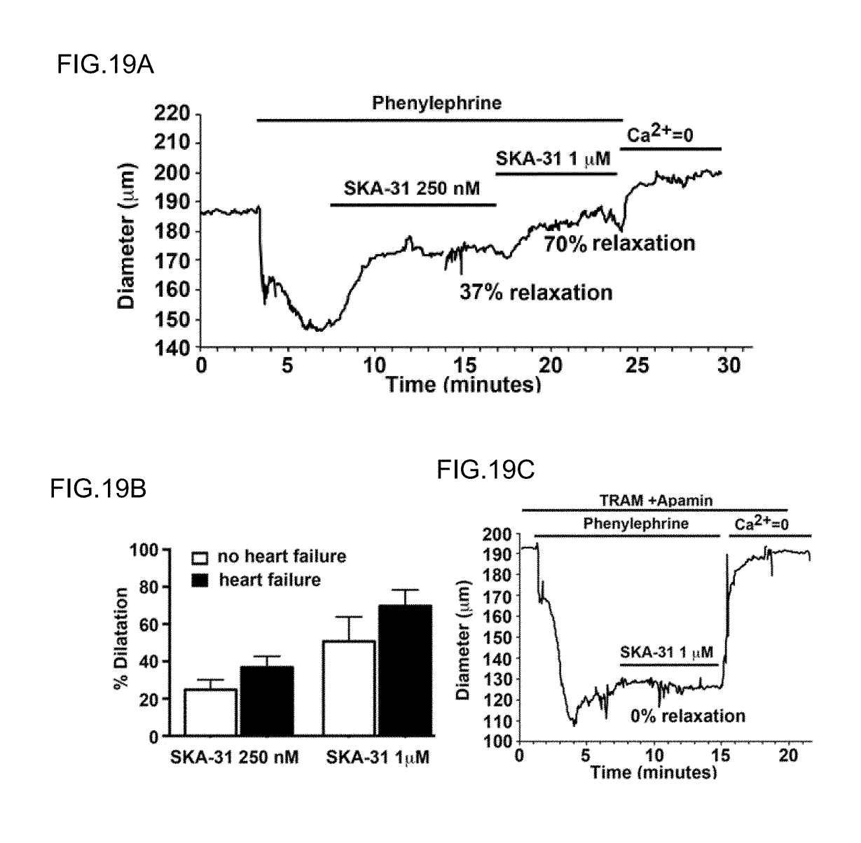

Intraluminal infusion of either 250 nM or 1 μM SKA-31 effectively dilated the vessels from both control mice and mice with HF (see FIGS. 19A-19B), at concentrations that are achievable with systemic administration. Thus, the “machinery” necessary to pharmacologically enhance EDHF is intact and functional in HF. These effects are mediated by activation of SK and IK1 channels, as shown previously (Mishra et al. 2012, Sankaranarayanan et al. 2009, Radtke et al. 2013) and by experiments in which intraluminal infusion of apamin and TRAM-34, specific inhibitors of SK and IK1 channels, prevented SKA-31 (1 μM)-mediated vasodilation of phenylephrine-constricted mesenteric resistance vessels (see FIG. 19C).

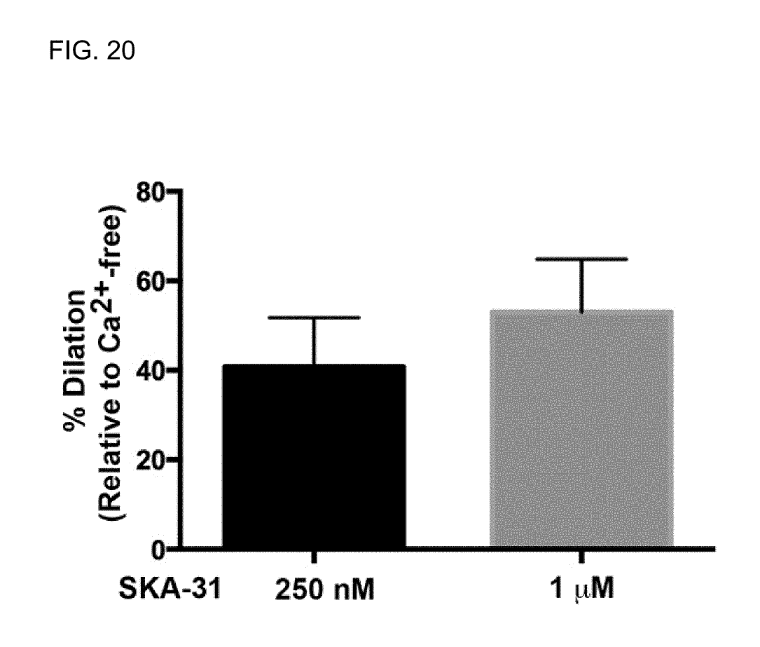

Augmentation of EDHF via activation of SK3 and IK1 channels was tested to see if it can relax phenylephrine-constricted mesenteric resistance vessels of BK β1 null mice, similar to the effects in WT mice. Perfusion of SKA-31 caused dilatation of vessels from β1 null mice to a similar extent as vessels from WT mice (see FIG. 20), indicating that BK channels are not required for EDHF-dilation.

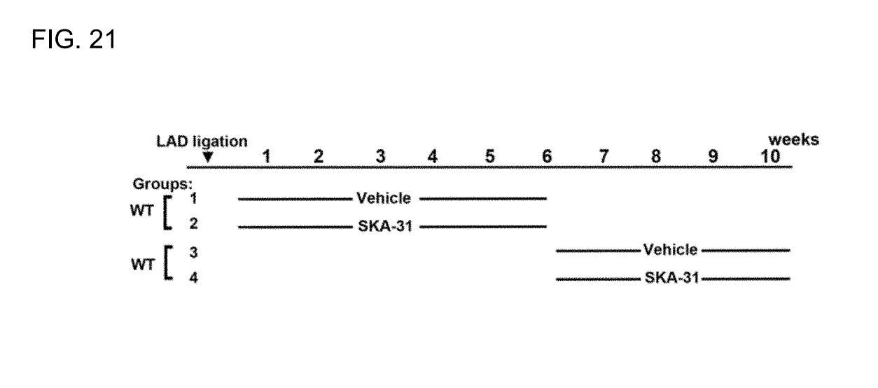

In vivo administration of SKA-31 may improve remodeling after MI. Two sets of experiments will be performed, one in which SKA-31 (30 mg/kg) or vehicle is administered IP daily to WT mice starting 72 hours after MI for 6 weeks, and the other in which SKA-31 is administered to WT mice with LV ejection fraction less than 40%, starting 6 weeks after LAD-ligation (see FIG. 21). 15 mice are in each of the four groups. LV function is followed weekly in a blinded fashion. Post-mortem, the severity of HF is inferred from lung weight, heart weight and body weight ratios, and serum markers. Blood pressure is also measured via tail cuff twice weekly. SKA-31 has been administered via IP injections to mice, with an adequate free SKA-31 level (above the EC₅₀ value for IK1 activation) 24 hours after IP injection of 10 mg/kg (Sankaranarayanan et al. 2009). To ensure adequate free plasma concentration, 30 mg/kg is administered, which has been shown to be safe. To confirm appropriate drug levels of SKA-31 in mice with HF, serum levels are measured using LC/MS. If LV function is improved after SKA-31 administration, an additional five mice are studied in each group to assess whether activation of endothelial SK/IK channels alters the ratio of IS to AAR.

Pharmacologic activation of EDHF may improve survival after LAD-ligation in BK β1 null mice. 30 mg/kg SKA-31 is injected daily via IP injections for 3 days prior to LAD-ligation. On the third day, the LAD is ligated in SKA-31-treated and vehicle-injected animals. SKA-31 is continued for 1 week after MI. If survival is improved, the experiments are continued for up to 6 weeks (without continued SKA-31) to determine if LV function is improved. If survival is improved but LV function is not improved, another set of experiments is performed in which SKA-31 treatment is started before LAD-ligation and continued throughout the 6-week-protocol. The primary endpoint is mortality. Additional endpoints are: (a) Clinical severity of HF; (b) Histopathological analysis; (c) Cardiac function, assessed at 2 days and 1, 3, and 6 weeks after MI by echocardiography and by pressure-volume loops at 6 weeks after MI.

To facilitate these studies, SKA-31 is administered via Alzet osmotic pumps. There is conflicting data about the role of SK channels in either promoting or preventing atrial fibrillation, perhaps dependent upon the species (Ozgen et al. 2007, Diness et al. 2010, Ellinor et al. 2010). Most pertinent for the studies in mice is the finding that genetic ablation of SK2 promoted atrial arrhythmias (Li et al. 2009), suggesting that atrial arrhythmias in SKA-31 treated mice are not increased. SK1 and SK2 channels are down-regulated in the atrium of patients with atrial fibrillation (Yu et al. 2012). SK currents play little role in the regulation of the action potential in normal ventricles, but are up-regulated in epicardial cells of the failing rabbit ventricle (Chua et al. 2011). During rapid pacing or fibrillation in failing ventricles, increased Ca²⁺ may activate SK channels and shorten the action potential, increasing the risk of ventricular fibrillation. Thus, telemeters will be implanted (Morrow et al. 2011) to determine whether SKA-31 increases the incidence of arrhythmias in the mice with HF.

### Example 16

Afterload Reduction Improves Cardiac Work in an Animal with Systolic Heart Failure

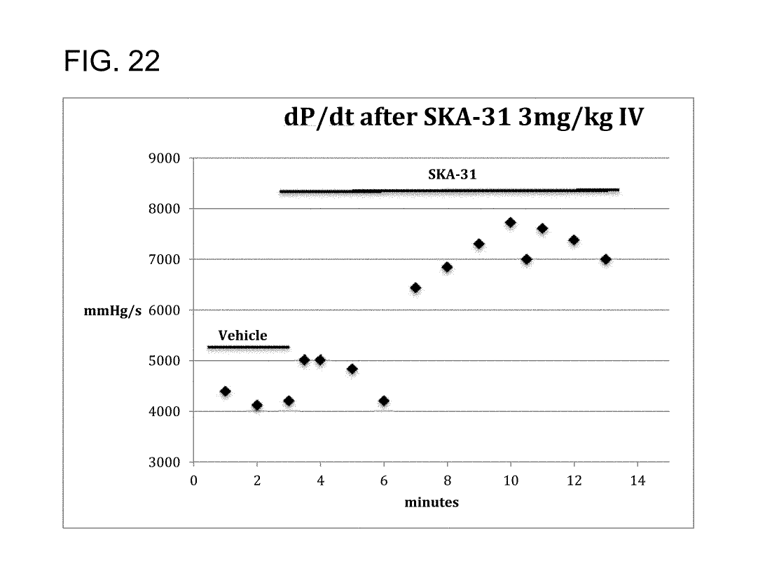

In this experiment, a wildtype mouse with heart failure (FAC 38%) was given an IV bolus of SKA-31 (3 mg/kg) through the left internal jugular vein during simultaneous recording of left ventricular pressure and volume using a Milar conductance catheter inserted through the right carotid artery. (FIG. 22)

BK channel deficient mice and wildtype littermates developed an ischemic cardiomyopathy after surgical ligation of the left anterior descending coronary artery. Systolic heart failure was confirmed six weeks later by noting a fractional area of change (FAC) of 40% or lower on 2D echocardiography (Visualsonics Vevo 2100). Mice with heart failure were laid supine on a heated pad under isoflurane anesthesia and mechanically depilated. The left internal jugular vein was isolated by blunt dissection and cannulated with a custom-built catheter made from PET tubing (McMaster). Blood loss and insensible losses were treated with a slow infusion of NS (0.1-0.5 ml) intravenously and/or subcutaneously. The right common carotid artery was isolated by blunt dissection and a 1 Fr conductance catheter (PVR-1045 made by Milar) was introduced and carefully advanced into the left ventricle (LV). Continuous LV pressure and volume measurements were recorded (MPVS Ultra by Milar with PowerLab 4/35 and LabChart by ADInstruments). After stable baseline LV measurements were recorded, vehicle (Cremophor EL made by Calbiochem) was infused intravenously into the cannulated internal jugular vein as a single bolus in the same volume later used to deliver an intravenous bolus of SKA-31 (Tocris) at 3 mg/kg. Volume measurements were calibrated by administration of 0.1 ml hypertonic saline and with blood obtained from cardiac puncture in a standardized cuvette (Milar).

As seen in FIG. 22, afterload reduction improves cardiac work in an animal with systolic heart failure. Without being limited by theory, this result is likely mediated by SKA-31 driven hyperpolarization and relaxation of vascular smooth muscle.

### DOCUMENTS

Ali, A. A., et al., Rat model of veno-arterial extracorporeal membrane oxygenation. J Transl Med, 2014. 12: p. 37.
Alioua, A., et al., Coupling of c-Src to large conductance voltage- and Ca2+-activated K+ channels as a new mechanism of agonist-induced vasoconstriction. Proc Natl Acad Sci USA, 2002. 99(22): p. 14560-5.
Amberg, G. C. and L. F. Santana, Downregulation of the BK channel beta1 subunit in genetic hypertension. Circ Res, 2003. 93(10): p. 965-71.
Amberg, G. C., et al., Modulation of the molecular composition of large conductance, Ca(2+) activated K(+) channels in vascular smooth muscle during hypertension. J Clin Invest, 2003. 112(5): p. 717-24.
Ambroisine, M. L., et al., Aldosterone-induced coronary dysfunction in transgenic mice involves the calcium-activated potassium (BKCa) channels of vascular smooth muscle cells. Circulation, 2007. 116(21): p. 2435-43.
Babu, G. J., et al., Ablation of sarcolipin enhances sarcoplasmic reticulum calcium transport and atrial contractility. Proc Natl Acad Sci USA, 2007. 104(45): p. 17867-72.
Baek, E. B. and S. J. Kim, Mechanisms of myogenic response: Ca(2+)-dependent and -independent signaling. J Smooth Muscle Res, 2011. 47(2): p. 55-65.
Bagi, Z., et al., Type 2 diabetic mice have increased arteriolar tone and blood pressure: enhanced release of COX-2-derived constrictor prostaglandins. Arteriosclerosis, thrombosis, and vascular biology, 2005. 25(8): p. 1610-6.
Bang, L., et al., Hydralazine-induced vasodilation involves opening of high conductance Ca2+-activated K+ channels. European journal of pharmacology, 1998. 361(1): p. 43-9.
Bao, L. and D. H. Cox, Gating and ionic currents reveal how the BKCa channel's Ca2+ sensitivity is enhanced by its beta1 subunit. J Gen Physiol, 2005. 126(4): p. 393-412.
Bayliss, W. M., On the local reactions of the arterial wall to changes of internal pressure. J Physiol, 1902. 28(3): p. 220-31.
Behrens, R., et al., hKCNMB3 and hKCNMB4, cloning and characterization of two members of the large-conductance calcium-activated potassium channel beta subunit family. FEBS Lett, 2000. 474(1): p. 99-106.
Benjamini, Y. and Y. Hochberg, Controlling the false discovery rate: a practical and powerful approach to multiple testing. JRSSB, 1995. 57: p. 289-300.
Bentzen, B. H., et al., The small molecule NS11021 is a potent and specific activator of Ca2+-activated big-conductance K+ channels. Molecular pharmacology, 2007. 72(4): p. 1033-44.
Bernal-Mizrachi, C., et al., Vascular respiratory uncoupling increases blood pressure and atherosclerosis. Nature, 2005. 435(7041): p. 502-6.
Bhan, A., et al., High-frequency speckle tracking echocardiography in the assessment of left ventricular function and remodeling after murine myocardial infarction. Am J Physiol Heart Circ Physiol, 2014. 306(9): p. H1371-83.
Borbouse, L., et al., Impaired function of coronary BK(Ca) channels in metabolic syndrome. American journal of physiology. Heart and circulatory physiology, 2009. 297(5): p. H1629-37.
Borchert, G. H., C. Yang, and F. Kolar, Mitochondrial BKCa channels contribute to protection of cardiomyocytes isolated from chronically hypoxic rats. American journal of physiology. Heart and circulatory physiology, 2011. 300(2): p. H507-13.
Brenner, R., et al., Vasoregulation by the beta1 subunit of the calcium-activated potassium channel. Nature, 2000. 407(6806): p. 870-6.
Britton, F. C., et al., Comparison of the properties of CLCA1 generated currents and I(Cl(Ca)) in murine portal vein smooth muscle cells. The Journal of physiology, 2002. 539(Pt 1): p. 107-17.
Brondum, E., et al., NS309 restores EDHF-type relaxation in mesenteric small arteries from type 2 diabetic ZDF rats. British journal of pharmacology, 2010. 159(1): p. 154-65.
Bulley, S., et al., TMEM16A/ANO1 channels contribute to the myogenic response in cerebral arteries. Circ Res, 2012. 111(8): p. 1027-36.
Bychkov, R., et al., Calcium-activated potassium channels and nitrate-induced vasodilation in human coronary arteries. J Pharmacol Exp Ther, 1998. 285(1): p. 293-8.
Caputo, A., et al., TMEM16A, a membrane protein associated with calcium-dependent chloride channel activity. Science, 2008. 322(5901): p. 590-4.
Choi, C., et al., cGMP-dependent protein kinase and the regulation of vascular smooth muscle cell gene expression: possible involvement of Elk-1 sumoylation. Am J Physiol Heart Circ Physiol, 2010. 299(5): p. H1660-70.
Christensen, K. L. and M. J. Mulvany, Mesenteric arcade arteries contribute substantially to vascular resistance in conscious rats. Journal of vascular research, 1993. 30(2): p. 73-9.
Chua, S. K., et al., Small-conductance calcium-activated potassium channel and recurrent ventricular fibrillation in failing rabbit ventricles. Circ Res, 2011. 108(8): p. 971-9.
Clements, R. T., et al., Rottlerin Increases Cardiac Contractile Performance and Coronary Perfusion Through BKCa++ Channel Activation After Cold Cardioplegic Arrest in Isolated Hearts. Circulation, 2011. 124(11 Suppl): p. S55-61.
Cox, D. H. and R. W. Aldrich, Role of the beta1 subunit in large-conductance Ca(2+)-activated K(+) channel gating energetics. Mechanisms of enhanced Ca(2+) sensitivity. J Gen Physiol, 2000. 116(3): p. 411-32.
Davis, A. J., et al., Potent vasorelaxant activity of the TMEM16A inhibitor T16A(inh)-A01. British journal of pharmacology, 2012.
Diness, J. G., et al., Inhibition of small-conductance Ca2+-activated K+ channels terminates and protects against atrial fibrillation. Circ Arrhythm Electrophysiol, 2010. 3(4): p. 380-90.
Dong, L., et al., Rotenone partially reverses decreased BK Ca currents in cerebral artery smooth muscle cells from streptozotocin-induced diabetic mice. Clinical and experimental pharmacology & physiology, 2009. 36(10): p. e57-64.
Dong, L., et al., Functional and molecular evidence for impairment of calcium-activated potassium channels in type-1 diabetic cerebral artery smooth muscle cells. Journal of cerebral blood flow and metabolism: official journal of the International Society of Cerebral Blood Flow and Metabolism, 2008. 28(2): p. 377-86.
Duvvuri, U., et al., TMEM16A induces MAPK and contributes directly to tumorigenesis and cancer progression. Cancer Res, 2012. 72(13): p. 3270-81.
Earley, S. and J. E. Brayden, Transient receptor potential channels and vascular function. Clinical science, 2010. 119(1): p. 19-36.
Earley, S., et al., TRPV4-dependent dilation of peripheral resistance arteries influences arterial pressure. American journal of physiology. Heart and circulatory physiology, 2009. 297(3): p. H1096-102.
Earley, S., et al., TRPV4 forms a novel Ca2+ signaling complex with ryanodine receptors and BKCa channels. Circulation research, 2005. 97(12): p. 1270-9.
Edwards, G. and A. H. Weston, Endothelium-derived hyperpolarizing factor—a critical appraisal. Prog Drug Res, 1998. 50: p. 107-33.
Edwards, G., et al., K+ is an endothelium-derived hyperpolarizing factor in rat arteries. Nature, 1998. 396(6708): p. 269-72.
Ellinor, P. T., et al., Common variants in KCNN3 are associated with lone atrial fibrillation. Nature genetics, 2010. 42(3): p. 240-4.
Evanson, K. W., et al., LRRC26 is a functional BK channel auxiliary gamma subunit in arterial smooth muscle cells. Circ Res, 2014. 115(4): p. 423-31.
Feletou, M., Calcium-activated potassium channels and endothelial dysfunction: therapeutic options? British journal of pharmacology, 2009. 156(4): p. 545-62.
Fernandez-Fernandez, J. M., et al., Gain-of-function mutation in the KCNMB1 potassium channel subunit is associated with low prevalence of diastolic hypertension. J Clin Invest, 2004. 113(7): p. 1032-9.
Fernandez-Tenorio, M., et al., Metabotropic regulation of RhoA/Rho-associated kinase by L-type Ca2+ channels: new mechanism for depolarization-evoked mammalian arterial contraction. Circ Res, 2011. 108(11): p. 1348-57.
Fraccarollo, D., et al., Improvement in left ventricular remodeling by the endothelial nitric oxide synthase enhancer AVE9488 after experimental myocardial infarction. Circulation, 2008. 118(8): p. 818-27.
Gao, Y., et al., IL-1beta modulate the Ca(2+)-activated big-conductance K channels (BK) via reactive oxygen species in cultured rat aorta smooth muscle cells. Molecular and cellular biochemistry, 2010. 338(1-2): p. 59-68.
Goldklang, M. P., et al., Treatment of experimental asthma using a single small molecule with anti-inflammatory and BK channel-activating properties. FASEB J, 2013.
Graham, L. N., et al., Sympathetic drive in anterior and inferior uncomplicated acute myocardial infarction. Circulation, 2004. 109(19): p. 2285-9.
Grimm, P. R., et al., Identification and localization of BK-beta subunits in the distal nephron of the mouse kidney. American journal of physiology. Renal physiology, 2007. 293(1): p. F350-9.
Gschwendt, M., et al., Rottlerin, a novel protein kinase inhibitor. Biochem Biophys Res Commun, 1994. 199(1): p. 93-8.
Gupta, D., et al., Nitrate Therapy for Heart Failure: Benefits and Strategies to Overcome Tolerance. JACC: Heart Failure, 2013. 183-191.
Hartzell, H. C., et al., Anoctamin/TMEM16 family members are Ca2+-activated Cl− channels. The Journal of physiology, 2009. 587(Pt 10): p. 2127-39.
He, Q., et al., Activation of the basolateral membrane Cl− conductance essential for electrogenic K+ secretion suppresses electrogenic Cl− secretion. Exp Physiol, 2011. 96(3): p. 305-16.
Hill, M. A., et al., Therapeutic potential of pharmacologically targeting arteriolar myogenic tone. Trends in pharmacological sciences, 2009. 30(7): p. 363-74.
Hill, M. A., et al., Arteriolar myogenic signalling mechanisms: Implications for local vascular function. Clinical hemorheology and microcirculation, 2006. 34(1-2): p. 67-79.
Irizarry, R. A., et al., Exploration, normalization, and summaries of high density oligonucleotide array probe level data. Biostatistics, 2003. 4(2): p. 249-64.
Jiang, Z., et al., Human and rodent MaxiK channel beta-subunit genes: cloning and characterization. Genomics, 1999. 55(1): p. 57-67.
Johnson, P. C., Autoregulation of blood flow. Circulation research, 1986. 59(5): p. 483-95.
Jones, S. P., et al., Endothelial nitric oxide synthase overexpression attenuates congestive heart failure in mice. Proceedings of the National Academy of Sciences of the United States of America, 2003. 100(8): p. 4891-6.
Jordan, J., Sluggish genes and hypertension. Journal of hypertension, 2008. 26(11): p. 2093-5.
Katz, S. D., et al., Impaired endothelium-mediated vasodilation in the peripheral vasculature of patients with congestive heart failure. Journal of the American College of Cardiology, 1992. 19(5): p. 918-25.
Knaus, H. G., et al., Primary sequence and immunological characterization of beta-subunit of high conductance Ca(2+)-activated K+ channel from smooth muscle. J Biol Chem, 1994. 269(25): p. 17274-8.
Knot, H. J. and M. T. Nelson, Regulation of arterial diameter and wall [Ca2+] in cerebral arteries of rat by membrane potential and intravascular pressure. The Journal of physiology, 1998. 508 (Pt 1): p. 199-209.
Kokubo, Y., et al., Association analysis between hypertension and CYBA, CLCNKB, and KCNMB1 functional polymorphisms in the Japanese population—the Suita Study. Circulation journal: official journal of the Japanese Circulation Society, 2005. 69(2): p. 138-42.
Kumar, D., et al., Distinct mouse coronary anatomy and myocardial infarction consequent to ligation. Coronary artery disease, 2005. 16(1): p. 41-4.
Lagaud, G. J., et al., Influence of type II diabetes on arterial tone and endothelial function in murine mesenteric resistance arteries. Journal of vascular research, 2001. 38(6): p. 578-89.
Large, W. A. and Q. Wang, Characteristics and physiological role of the Ca(2+)-activated Cl− conductance in smooth muscle. The American journal of physiology, 1996. 271(2 Pt 1): p. C435-54.
Larsen, B. T., et al., Epoxyeicosatrienoic and dihydroxyeicosatrienoic acids dilate human coronary arterioles via BK(Ca) channels: implications for soluble epoxide hydrolase inhibition. American journal of physiology. Heart and circulatory physiology, 2006. 290(2): p. H491-9.
Layne, J. J., et al., NFATc3 regulates BK channel function in murine urinary bladder smooth muscle. American journal of physiology. Cell physiology, 2008. 295(3): p. C611-23.
Leo, M. D., et al., Dynamic regulation of beta1 subunit trafficking controls vascular contractility. Proc Natl Acad Sci USA, 2014. 111(6): p. 2361-6.
Levesque, M. J., J. F. Cornhill, and R. M. Nerem, Vascular casting. A new method for the study of the arterial endothelium. Atherosclerosis, 1979. 34(4): p. 457-67.
Li, N., et al., Ablation of a Ca2+-activated K+ channel (SK2 channel) results in action potential prolongation in atrial myocytes and atrial fibrillation. J Physiol, 2009. 587(Pt 5): p. 1087-100.
Liu, G., et al., Assembly of a Ca(2+)-dependent BK channel signaling complex by binding to beta2 adrenergic receptor. Embo J, 2004. 23(11): p. 2196-205.
Liu, Y. and D. D. Gutterman, Vascular control in humans: focus on the coronary microcirculation. Basic research in cardiology, 2009. 104(3): p. 211-27.
Liu, Y., et al., H2O2 is the transferable factor mediating flow-induced dilation in human coronary arterioles. Circulation research, 2011. 108(5): p. 566-73.
Long, X., et al., The smooth muscle cell-restricted KCNMB1 ion channel subunit is a direct transcriptional target of serum response factor and myocardin. The Journal of biological chemistry, 2009. 284(48): p. 33671-82.
Lu, T., et al., Impaired Ca2+-dependent activation of large-conductance Ca2+-activated K+ channels in the coronary artery smooth muscle cells of Zucker Diabetic Fatty rats. Biophysical journal, 2008. 95(11): p. 5165-77.
Lu, T., et al., Regulation of coronary arterial BK channels by caveolae-mediated angiotensin II signaling in diabetes mellitus. Circulation research, 2010. 106(6): p. 1164-73.
Lymperopoulos, A., et al., Reduction of sympathetic activity via adrenal-targeted GRK2 gene deletion attenuates heart failure progression and improves cardiac function after myocardial infarction. The Journal of biological chemistry, 2010. 285(21): p. 16378-86.
Mack, C. P., Signaling mechanisms that regulate smooth muscle cell differentiation. Arterioscler Thromb Vasc Biol, 2011. 31(7): p. 1495-505.
Mahida, S. and P. T. Ellinor, New advances in the genetic basis of atrial fibrillation. J Cardiovasc Electrophysiol, 2012. 23(12): p. 1400-6.
Manoury, B., A. Tamuleviciute, and P. Tammaro, TMEM16A/anoctamin 1 protein mediates calcium-activated chloride currents in pulmonary arterial smooth muscle cells. The Journal of physiology, 2010. 588(Pt 13): p. 2305-14.
Matchkov, V. V., et al., Bestrophin-3 (vitelliform macular dystrophy 2-like 3 protein) is essential for the cGMP-dependent calcium-activated chloride conductance in vascular smooth muscle cells. Circulation research, 2008. 103(8): p. 864-72.
McAlpine, H. M. and S. M. Cobbe, Neuroendocrine changes in acute myocardial infarction. Am J Med, 1988. 84(3A): p. 61-6.
McGahon, M. K., et al., Diabetes downregulates large-conductance Ca2+-activated potassium beta 1 channel subunit in retinal arteriolar smooth muscle. Circulation research, 2007. 100(5): p. 703-11.
McGahon, M. K., et al., Selective downregulation of the BKbeta1 subunit in diabetic arteriolar myocytes. Channels, 2007. 1(3): p. 141-3.
Meredith, A. L., et al., BK calcium-activated potassium channels regulate circadian behavioral rhythms and pacemaker output. Nature neuroscience, 2006. 9(8): p. 1041-9.
Michael, L. H., et al., Myocardial ischemia and reperfusion: a murine model. The American journal of physiology, 1995. 269(6 Pt 2): p. H2147-54.
Mishra, R. C., et al., SKA-31, a novel activator of SKCa and IKCa channels, increases coronary flow in male and female rat hearts. Cardiovascular research, 2012.
Moessler, H., et al., The SM 22 promoter directs tissue-specific expression in arterial but not in venous or visceral smooth muscle cells in transgenic mice. Development, 1996. 122(8): p. 2415-25.
Moon, C., et al., Erythropoietin reduces myocardial infarction and left ventricular functional decline after coronary artery ligation in rats. Proc Natl Acad Sci USA, 2003. 100(20): p. 11612-7.
Morita, T. and K. Hayashi, Arp5 is a key regulator of myocardin in smooth muscle cells. J Cell Biol, 2014. 204(5): p. 683-96.
Morrow, J. P., et al., Mice with cardiac overexpression of peroxisome proliferator-activated receptor gamma have impaired repolarization and spontaneous fatal ventricular arrhythmias. Circulation, 2011. 124(25): p. 2812-21.
Mroz, M. S. and S. J. Keely, Epidermal growth factor chronically upregulates Ca(2+)-dependent Cl(−) conductance and TMEM16A expression in intestinal epithelial cells. J Physiol, 2012. 590(Pt 8): p. 1907-20.
Navedo, M. F., et al., Ca(v)1.3 channels produce persistent calcium sparklets, but Ca(v)1.2 channels are responsible for sparklets in mouse arterial smooth muscle. American journal of physiology. Heart and circulatory physiology, 2007. 293(3): p. H1359-70.
Nelson, M. T., et al., Relaxation of arterial smooth muscle by calcium sparks. Science, 1995. 270(5236): p. 633-7.
Nieves-Cintron, M., et al., Activation of NFATc3 down-regulates the beta1 subunit of large conductance, calcium-activated K+ channels in arterial smooth muscle and contributes to hypertension. The Journal of biological chemistry, 2007. 282(5): p. 3231-40.
Nieves-Cintron, M., et al., The control of Ca2+ influx and NFATc3 signaling in arterial smooth muscle during hypertension. Proceedings of the National Academy of Sciences of the United States of America, 2008. 105(40): p. 15623-8.
Nystoriak, M. A., et al., AKAP150 contributes to enhanced vascular tone by facilitating large-conductance Ca2+-activated K+ channel remodeling in hyperglycemia and diabetes mellitus. Circ Res, 2014. 114(4): p. 607-15.
Ohya, S., et al., Cardioprotective effects of estradiol include the activation of large-conductance Ca(2+)-activated K(+) channels in cardiac mitochondria. American journal of physiology. Heart and circulatory physiology, 2005. 289(4): p. H1635-42.
Ozgen, N., et al., Early electrical remodeling in rabbit pulmonary vein results from trafficking of intracellular SK2 channels to membrane sites. Cardiovascular research, 2007. 75(4): p. 758-69.
Phillips, S. A., F. A. Sylvester, and J. C. Frisbee, Oxidant stress and constrictor reactivity impair cerebral artery dilation in obese Zucker rats. American journal of physiology. Regulatory, integrative and comparative physiology, 2005. 288(2): p. R522-30.
Pluger, S., et al., Mice with disrupted BK channel beta1 subunit gene feature abnormal Ca(2+) spark/STOC coupling and elevated blood pressure. Circulation research, 2000. 87(11): p. E53-60.
Pluznick, J. L., et al., BK-{beta}1 subunit: immunolocalization in the mammalian connecting tubule and its role in the kaliuretic response to volume expansion. American journal of physiology. Renal physiology, 2005. 288(4): p. F846-54.
Pyott, S. J., et al., Cochlear function in mice lacking the BK channel alpha, beta1, or beta4 subunits. The Journal of biological chemistry, 2007. 282(5): p. 3312-24.
Radtke, J., et al., Activation of KCa 3.1 by SKA-31 induces arteriolar dilatation and lowers blood pressure in normo- and hypertensive connexin40-deficient mice. British journal of pharmacology, 2013. 170(2): p. 293-303.
Redfors, B., Y. Shao, and E. Omerovic, Myocardial infarct size and area at risk assessment in mice. Exp Clin Cardiol, 2012. 17(4): p. 268-72.
Rusch, N. J., BK channels in cardiovascular disease: a complex story of channel dysregulation. American journal of physiology. Heart and circulatory physiology, 2009. 297(5): p. H1580-2.
Sakamoto, K., et al., A novel opener of large-conductance Ca2+-activated K+(BK) channel reduces ischemic injury in rat cardiac myocytes by activating mitochondrial K(Ca) channel. Journal of pharmacological sciences, 2008. 108(1): p. 135-9.
Sankaranarayanan, A., et al., Naphtho[1,2-d]thiazol-2-ylamine (SKA-31), a new activator of KCa2 and KCa3.1 potassium channels, potentiates the endothelium-derived hyperpolarizing factor response and lowers blood pressure. Molecular pharmacology, 2009. 75(2): p. 281-95.
Sato, T., et al., Mitochondrial Ca2+-activated K+ channels in cardiac myocytes: a mechanism of the cardioprotective effect and modulation by protein kinase A. Circulation, 2005. 111(2): p. 198-203.
Schrier, R. W. and W. T. Abraham, Hormones and hemodynamics in heart failure. The New England journal of medicine, 1999. 341(8): p. 577-85.
Schroeder, B. C., et al., Expression cloning of TMEM16A as a calcium-activated chloride channel subunit. Cell, 2008. 134(6): p. 1019-29.
Schubert, R. and M. T. Nelson, Protein kinases: tuners of the BKCa channel in smooth muscle. Trends Pharmacol Sci, 2001. 22(10): p. 505-12.
Senti, M., et al., Protective effect of the KCNMB1 E65K genetic polymorphism against diastolic hypertension in aging women and its relevance to cardiovascular risk. Circulation research, 2005. 97(12): p. 1360-5.
Sigurdsson, A., P. Held, and K. Swedberg, Short- and long-term neurohormonal activation following acute myocardial infarction. American heart journal, 1993. 126(5): p. 1068-76.
Singh, H., et al., mitoBKCa is encoded by the Kcnma1 gene, and a splicing sequence defines its mitochondrial location. Proc Natl Acad Sci USA, 2013. 110(26): p. 10836-41.
Smith, J. G., et al., Impact of Ancestry and Common Genetic Variants on QT Interval in African Americans. Circ Cardiovasc Genet, 2012. 5(6): p. 647-655.
Soltoff, S. P., Rottlerin is a mitochondrial uncoupler that decreases cellular ATP levels and indirectly blocks protein kinase Cdelta tyrosine phosphorylation. J Biol Chem, 2001. 276(41): p. 37986-92.
Soltoff, S. P., Rottlerin: an inappropriate and ineffective inhibitor of PKCdelta. Trends Pharmacol Sci, 2007. 28(9): p. 453-8.
Sonkusare, S. K., et al., Elementary Ca2+ signals through endothelial TRPV4 channels regulate vascular function. Science, 2012. 336(6081): p. 597-601.
Takagawa, J., et al., Myocardial infarct size measurement in the mouse chronic infarction model: comparison of area- and length-based approaches. J Appl Physiol (1985), 2007. 102(6): p. 2104-11.
Taylor, A. L., et al., Combination of isosorbide dinitrate and hydralazine in blacks with heart failure. The New England journal of medicine, 2004. 351(20): p. 2049-57.
Taylor, A. L., et al., Early and sustained benefit on event-free survival and heart failure hospitalization from fixed-dose combination of isosorbide dinitrate/hydralazine: consistency across subgroups in the African-American Heart Failure Trial. Circulation, 2007. 115(13): p. 1747-53.
Taylor, M. S., et al., Altered expression of small-conductance Ca2+-activated K+ (SK3) channels modulates arterial tone and blood pressure. Circulation research, 2003. 93(2): p. 124-31.
Thomas-Gatewood, C., et al., TMEM16A channels generate Ca(2)(+)-activated Cl(−) currents in cerebral artery smooth muscle cells. American journal of physiology. Heart and circulatory physiology, 2011. 301(5): p. H1819-27.
Tomas, M., et al., Genetic variation in the KCNMA1 potassium channel alpha subunit as risk factor for severe essential hypertension and myocardial infarction. Journal of hypertension, 2008. 26(11): p. 2147-53.
Tomaselli, G. F. and D. P. Zipes, What causes sudden death in heart failure? Circ Res, 2004. 95(8): p. 754-63.
Via, M., et al., E65 K polymorphism in KCNMB1 gene is not associated with ischaemic heart disease in Spanish patients. Journal of human genetics, 2005. 50(11): p. 604-6.
Wan, E., et al., Reduced vascular smooth muscle BK channel current underlies heart failure-induced vasoconstriction in mice. FASEB J, 2013.
Wang, X., et al., Essential role of mitochondrial Ca2+-activated and ATP-sensitive K+ channels in sildenafil-induced late cardioprotection. Journal of molecular and cellular cardiology, 2008. 44(1): p. 105-13.
West, J., et al., Pulmonary hypertension in transgenic mice expressing a dominant-negative BMPRII gene in smooth muscle. Circulation research, 2004. 94(8): p. 1109-14.
Wu, R. S., et al., Positions of beta2 and beta3 subunits in the large-conductance calcium- and voltage-activated BK potassium channel. The Journal of general physiology, 2013. 141(1): p. 105-17.
Wulf, H., et al., Molecular investigations of BK(Ca) channels and the modulatory beta-subunits in porcine basilar and middle cerebral arteries. J Mol Histol, 2009. 40(2): p. 87-97.
Xie, P., et al. i, CHIP represses myocardin-induced smooth muscle cell differentiation via ubiquitin-mediated proteasomal degradation. Mol Cell Biol, 2009. 29(9): p. 2398-408.
Xu, H., et al., Large-conductance Ca2+-activated K+ channel beta1-subunit knockout mice are not hypertensive. American journal of physiology. Heart and circulatory physiology, 2011. 300(2): p. H476-85.
Xu, W., et al., Cytoprotective role of Ca2+-activated K+ channels in the cardiac inner mitochondrial membrane. Science, 2002. 298(5595): p. 1029-33.
Yan, J. and R. W. Aldrich, LRRC26 auxiliary protein allows BK channel activation at resting voltage without calcium. Nature, 2010. 466(7305): p. 513-6.
Yang, Y., et al., Function of BKCa Channels Is Reduced in Human Vascular Smooth Muscle Cells From Han Chinese Patients With Hypertension. Hypertension, 2012.
Yu, T., et al., Decreased expression of small-conductance Ca2+-activated K+ channels SK1 and SK2 in human chronic atrial fibrillation. Life sciences, 2012. 90(5-6): p. 219-27.
Zakharov, S. I., et al., Activation of the BK (SLO1) potassium channel by mallotoxin. J Biol Chem, 2005. 280(35): p. 30882-7.
Zelis, R. and S. F. Flaim, Alterations in vasomotor tone in congestive heart failure. Progress in cardiovascular diseases, 1982. 24(6): p. 437-59.
Zhang, D. M., et al., Muscle-specific f-box only proteins facilitate bk channel beta(1) subunit downregulation in vascular smooth muscle cells of diabetes mellitus. Circulation research, 2010. 107(12): p. 1454-9.
Zhou, X. B., et al., Dual role of protein kinase C on BK channel regulation. Proceedings of the National Academy of Sciences of the United States of America, 2010. 107(17): p. 8005-10.

All patents, patent applications, and publications cited above are incorporated herein by reference in their entirety as if recited in full herein.

The invention being thus described, it will be obvious that the same may be varied in many ways. Such variations are not to be regarded as a departure from the spirit and scope of the invention and all such modifications are intended to be included within the scope of the following claims.

What is claimed is:

1. A compound having formula (I): 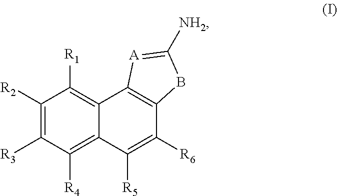
wherein
A and B are independently selected from the group consisting of S or N;
R₁, R₂, R₃, R₄, and R₆ are independently selected from the group consisting of H, halogen, alkyl, ester, ether, thioether, aryl, heteroaryl, CN, NO₂, and amine;
R₅, is selected from the group consisting of H, ester, thioether, aryl, heteroaryl, NO₂, and amine;
wherein each alkyl is optionally substituted with a group consisting of halide, ether, and combinations thereof;
wherein each aryl and each heteroaryl are optionally substituted with a group consisting of halide, ether, C₁₋₄alkyl, and combinations thereof; and
wherein each amine is optionally substituted with a group selected from halide, C₁₋₄alkyl, and combinations thereof,
and crystalline forms, hydrates, or pharmaceutically acceptable salts thereof,
with the provisio that the compound is not

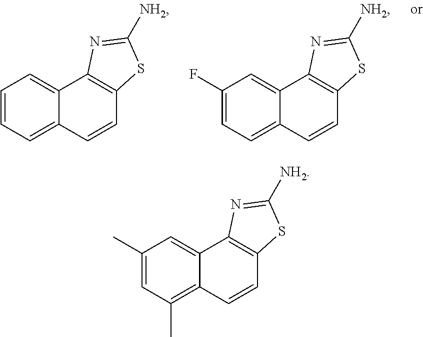

2. The compound according to claim 1, wherein the compound has the formula (II) 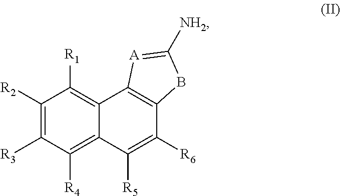
wherein
A and B are independently selected from the group consisting of S or N;
R₁, R₂, R₃, R₄, and R₆ are independently selected from the group consisting of H, halogen, C₁₋₆alkyl, —X—C₁₋₆alkyl, CN, —NO₂, —C(O)—R₇ and —N(R₇)(R₈);
R₅, is selected from the group consisting of H, —NO₂, —C(O)—R₇ and N(R₇)(R₈);
wherein X is independently selected from the group consisting of S or O;
wherein Y is selected from the group consisting of no atom, S or O; and
R₇ and R₈ are independently selected from the group consisting of H, halogen, C₁₋₆alkyl, and CN,
and crystalline forms, hydrates, or pharmaceutically acceptable salts thereof.

3. The compound according to claim 1, which is selected from the group consisting of 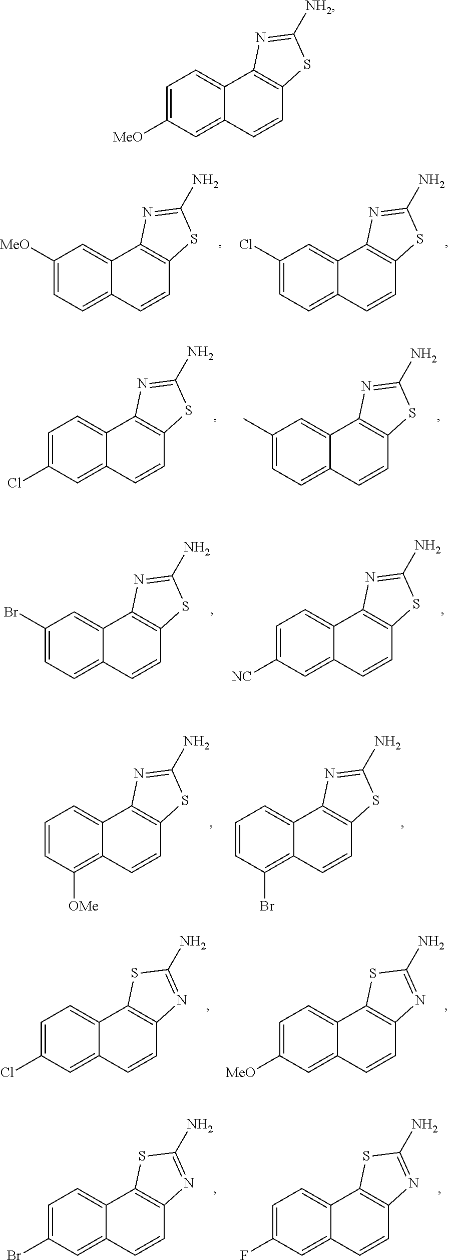
and crystalline forms, hydrates, or pharmaceutically acceptable salts thereof.

4. A compound having the structure: 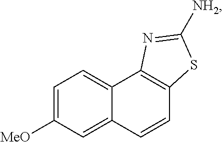 [SMILES: COc1ccc2c(ccc3sc(N)nc32)c1]
or a crystalline form, hydrate, or pharmaceutically acceptable salt thereof.

5. A compound having the structure: 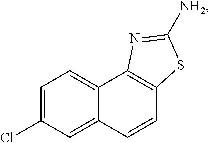 [SMILES: Nc1nc2c(ccc3cc(Cl)ccc32)s1]
or a crystalline form, hydrate, or pharmaceutically acceptable salt thereof.

6. A pharmaceutical composition comprising a pharmaceutically acceptable carrier and a compound according to claim 1.

7. The pharmaceutical composition of claim 6, further comprising one or more additional active agents.

8. The pharmaceutical composition of claim 7, wherein the one or more additional active agents are selected from the group consisting of nitrates, angiotensin-converting enzyme (ACE) inhibitors, angiotensin receptor blockers, beta-adrenergic blockers, and aldosterone receptor antagonists.

9. The pharmaceutical composition of claim 8, wherein the nitrates are selected from the group consisting of nitroglycerin, isorbide mononitrate, isosorbide dinitrate, pentaerythrityl tetranitrate, sodium nitroprusside, molsidomine, SIN-1, and combinations thereof.

10. The pharmaceutical composition of claim 8, wherein the ACE inhibitors are selected from the group consisting of alacepril, alatriopril, altiopril calcium, ancovenin, benazepril, benazepril hydrochloride, benazeprilat, benzoylcaptopril, captopril, captopril-cysteine, captopril-glutathione, ceranapril, ceranopril, ceronapril, cilazapril, cilazaprilat, delapril, delapril-diacid, enalapril, enalaprilat, enapril, epicaptopril, foroxymithine, fosfenopril, fosenopril, fosenopril sodium, fosinopril, fosinopril sodium, fosinoprilat, fosinoprilic acid, glycopril, hemorphin-4, idrapril, imidapril, indolapril, indolaprilat, libenzapril, lisinopril, lyciumin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spiropril hydrochloride, temocapril, temocapril hydrochloride, teprotide, trandolapril, trandolaprilat, utibapril, zabicipril, zabiciprilat, zofenopril, zofenoprilat, casokinins, lactokinins, lactotripeptides (such as Val-Pro-Pro and Ile-Pro-Pro) and combinations thereof.

11. The pharmaceutical composition of claim 8, wherein the angiotensin receptor blockers are selected from the group consisting of candesartan, candesartan cilexetil, losartan, valsartan, irbesartan, tasosartan, telmisartan, eprosartan, L158,809, saralasin, olmesartan and combinations thereof.

12. The pharmaceutical composition of claim 8, wherein the beta-adrenergic blockers are selected from the group consisting of acebutolol, atenolol, betaxolol, bevantolol, bisoprolol, celiprolol, cetamolol, epanolol, esmolol, levobetaxolol, practolol, propranolol, bucindolol, carteolol, carvedilol, nadolol, oxyprenolol, penbutolol, pindolol, sotalol, timolol, metoprolol, nebivolol, butaxamine, IC-118,551, SR59230A, and combinations thereof.

13. The pharmaceutical composition of claim 8, wherein the aldosterone receptor antagonists are selected from the group consisting of spironolactone, eplerenone, canrenone, propenone, mexrenone, and combinations thereof.

14. A method for treating or ameliorating the effects of a condition in a subject in need thereof comprising administering to the subject an effective amount of a compound according to claim 1.

15. A method for treating or ameliorating the effects of a condition in a subject in need thereof comprising administering to the subject an effective amount of a pharmaceutical composition according to claim 6.

16. The method of claim 14, wherein the condition is heart failure syndrome.

17. The method of claim 14, wherein the condition is high blood pressure.

18. The method of claim 14, wherein the condition is diabetes.

19. The method according to claim 14, wherein the subject is a mammal.

20. The method according to claim 19, wherein the mammal is selected from a group consisting of humans, primates, farm animals, domestic animals and laboratory animals.

21. The method according to claim 19, wherein the mammal is a human.

22. A method for treating or ameliorating the effects of heart failure syndrome (HF) in a subject in need thereof comprising administering to the subject an effective amount of a pharmaceutical composition according to claim 6.

23. The method according to claim 22, wherein the subject is a mammal.

24. The method according to claim 23, wherein the mammal is selected from a group consisting of humans, primates, farm animals, domestic animals and laboratory animals.

25. The method according to claim 23, wherein the mammal is a human.

26. A kit for treating or ameliorating the effects of a condition in a subject in need thereof, the kit comprising an effective amount of a compound according to claim 1, packaged together with instructions for its use.

27. A kit for treating or ameliorating the effects of a condition in a subject in need thereof, the kit comprising an effective amount of a pharmaceutical composition according to claim 6, packaged together with instructions for its use.

28. The kit of claim 26, wherein the condition is heart failure syndrome.

29. The kit of claim 26, wherein the condition is high blood pressure.

30. The kit of claim 26, wherein the condition is diabetes.

31. A kit for treating or ameliorating the effects of heart failure syndrome (HF) in a subject in need thereof, the kit comprising an effective amount of a pharmaceutical composition according claim 6, packaged together with instructions for its use.

32. A composition comprising a compound according to claim 1.

33. The composition according to claim 32, which is a research reagent.
